(19) United States
(12) Patent Application Publication
(10) Pub. No.: US 2010/0170852 A1
(43) Pub. Date: Jul. 8, 2010

(54) Method and Device for Gravity Flow Chromatography

(76) Inventors: Chris Suh, San Jose, CA (US); Lee Hoang, Santa Clara, CA (US); Douglas T. Gjerde, Saratoga, CA (US)

Correspondence Address: PHYNEXUS, INC., 3670 CHARTER PARK DRIVE, SAN JOSE, CA, 95136, US

(21) Appl. No.: 12/729,103

(22) Filed: Mar. 22, 2010

Related U.S. Application Data
(63) Continuation-in-part of application No. 12/709,487, filed on Feb. 21, 2010.
Application No. 12/709,487 is a continuation-in-part of application No. 12/435,381, filed on May 4, 2009.
Application No. 12/435,381 is a continuation-in-part of application No. 11/292,707, filed on Dec. 1, 2005, now abandoned.
(60) Provisional application No. 60/632,966, filed on Dec. 3, 2004.

(51) Int. Cl. B01D 15/22

(52) U.S. Cl. 210/656; 210/198.2

(57) ABSTRACT

The invention provides gravity chromatographic columns for the purification of a material (e.g., a biological macromolecule, such as a peptide, protein or nucleic acid) from a sample solution, as well as methods for making and using such columns. The columns typically include a bed of media positioned above a bottom frit or between a bottom and top frit. In some embodiments, the columns employ modified pipette tips as column bodies. In some embodiments, the columns employ modified plates or racks as column bodies. In some embodiments, the invention provides methods and devices for gel filtration, desalting, buffer exchange, ion exchange, ion-pairing, normal phase and reverse phase chromatography. In some embodiments, the invention provides multiplexing gravity flow chromatography on a liquid handling robotic system.

### CROSS REFERENCE TO RELATED APPLICATIONS

This application is a continuation-in-part of U.S. application Ser. No. 12/709,487 filed Feb. 21, 2010, which is a continuation in part of U.S. application Ser. No. 12/435,381 filed May 4, 2009, which is a continuation-in-part of U.S. application Ser. No. 11/292,707 filed Dec. 1, 2005, now abandoned, which claims the benefit of Provisional U.S. Application No. 60/632,966 filed Dec. 3, 2004, the disclosure of each is incorporated herein by reference in its entirety for all purposes.

### FIELD OF THE INVENTION

This invention relates to methods and devices for using an automated, multiplexed, preparative type of liquid chromatography to treat, separate or prepare material or materials in a sample solution. This invention also relates to miniaturized gravity columns for manual use. The materials that are separated can include biomolecules, particularly biological macromolecules such as proteins, peptides and nucleic acids, and other materials of interest. The device and method of this invention are particularly useful for any type of aqueous based elution systems of chromatography including size exclusion chromatography, gel filtration chromatography, buffer exchange and desalting sample preparation, and affinity, ion exchange, salting out, hydrophobic interaction and aqueous normal phase chromatography. The device and method of this invention also are particularly useful in many types of organic solvent and aqueous based elution chromatography systems that contain some organic solvent including reverse phase chromatography and for chaotropic normal phase chromatography.

### BACKGROUND OF THE INVENTION

Preparative liquid chromatography is a powerful technology for separating, purifying or treating materials or substances including biomolecules. Preparative liquid chromatography is one of the primary tools used for preparing protein samples or nucleic acids samples prior to analysis by any of a variety of analytical techniques, including capillary electrophoresis, HPLC, mass spectrometry, surface plasmon resonance, nuclear magnetic resonance, x-ray crystallography, and the like, or biological assays including enzyme analysis, cell based assays or similar tests. It is often critical that interfering contaminants be removed from the sample and that the substance of interest is present at some minimum concentration. Thus, sample preparation methods are needed that permit the separation or treatment of small volume samples with minimal sample loss. In some cases, large amounts of purified materials may be needed which in turn may require larger and more concentrated starting sample volumes. This may require larger column beds to prevent overloading of the chromatographic system.

Providing an automated, multiplexed preparative method of liquid chromatography has been the subject of ongoing work for many years. Some of this work has involved operating in parallel several high performance liquid chromatographic (HPLC) instruments. While these instruments are effective in preparative preparation of materials, the cost of owning several instruments may be prohibitive. In addition, operating several instruments in parallel is complicated and labor intensive. Some newer HPLC instruments may contain several columns within one instrument that may be operated in parallel. But the instrument is still complex to purchase and operate and the type, size and capacity of the columns is limited.

Filter plates have been used in some automated extraction processes. 96-well filter plates containing extraction materials placed on top of the filter portion of the plate and are used in vacuum manifolds, centrifuges and robotic liquid handlers. These plates use vacuum to move liquids through the extraction material and exit the bottom of the plate. The plate may be moved from station to station in the robotic liquid handler to add sample, wash and collect the purified materials. Extraction processes employ high sample component affinity coefficients for the stationary phase and on-off type of separations. In these types of separations the component of interest sticks (adsorbs) to the stationary phase and the appropriate buffer or solvent conditions. When the buffer or solvent is changed, the component of interest un-sticks (desorbs) and moves quickly through the column. Air may enter the extraction phase of the plate without harm to the separation process. If too much airs enters one or more wells the vacuum may be lowered and prevent or disturb the extraction process. Filter plate extraction plates do not have the resolving power of a chromatographic column separation process. In addition, a filter plate that is operated by vacuum has less flexibility in the number of samples that can be processed at one time. Normally all wells of the plate have to be used simultaneously.

There is a need for a chromatographic system and columns that can be operated with a robotic liquid handler. However, chromatographic columns cannot have air introduced into the system. Air introduced into a column will produce fluid channeling in the column and will also change the backpressure of the column. Channeling in a chromatographic column destroys the resolving power of the column. Liquids flow around the air pockets in the column bed rather than through the entire bed thereby destroying flow path bed uniformity. Furthermore, a backpressure change would change the liquid flow rate through the column. The flow rate of fluid pumped through the chromatographic column must be controlled accurately and precisely to maintain chromatographic column performance and also to determine when to collect the faction of interest. Also, even if the average flow rate is known, the flow rate can change as the chromatographic process proceeds, making it difficult to determine when to collect the fraction of interest.

Another important issue with chromatography is the accurate injection or addition of sample material to the top of the column. An exact known volume of material has to be injected to maintain sample peak resolution. This may not be as important if the selectivity of the column for the sample material is very high. In these cases, the sample will bind to the top of the column in a tight band. But in cases where the selectivity is not high, the sample peak may spread upon injection and may be different from column-to-column if the injection of material is not done exactly the same with each column.

This invention provides an automated, multiplexed, preparative gravity column liquid chromatography apparatus and process that is operated with a robotic liquid handler. A plurality of packed bed columns cannot have the same backpressure to liquid flow for each column. The back pressures must vary from column to column. Gravity is constant. But since the gravity flow force is dependent on the amount of liquid above the column and is not a constant force, it is expected that gravity flow column flow rates would vary from column to column. Aliquots of liquid must be added to the top of the column at exactly the correct time. If the aliquot is added too late, the column runs dry and the separation is ruined. If the aliquot is added too early, the liquid from the previous aliquot is mixed with the aliquot from the new liquid and the separation is ruined. This makes coordination of the chromatographic steps conditioning, injection, chromatography, washing, and the elution of across a plate or rack of columns seem impossible. It would seem to be impossible to run even two columns in parallel. It would seem to impossible to run even one column in an automated robot by gravity flow impossible unless the flow conditions of the single column were measured ahead of time and then the robotic liquid handler was programmed to accommodate the single column. Even with one column, it still must be known the exact time to add each aliquot of liquid to the head of the column with out the column bed running dry or adding the aliquot too soon and mixing with the previous aliquot. This makes even manual operation of a column where liquid aliquots are added to the column with manual operation impractical. Each column is different and thus the flow is different from one column to the next column. The flow rate on a gravity flow column in an automated liquid handler is not monitored. Yet, if the method is timed and programmed, the addition of a liquid aliquot to the top of the gravity column must be done for all columns at the same time. There exists a need for automated or semi-automated gravity flow preparative liquid chromatography. The automated method must be able to reliably perform all steps of conditioning, injection, chromatography, washing, and the elution of the columns 1-96 at a time or 1-384 at a time.

### SUMMARY OF THE INVENTION

This invention provides a multiplexed, preparative gravity column liquid chromatography apparatus and process. The process can be automated or manual. The gravity columns have small diameters and can be operated with a 96-well 9.0 mm center-to-center format or 384-well 4.5 mm center-to-center format. For the 96-well rack or plate format, 1-96 columns are operated in parallel. For the 384-well rack or plate format, 1-384 columns can be operated in parallel. The columns used in the apparatus are manufactured to have similar backpressures and flow rates. A paused flow system of liquid aliquot addition is used to prevent the columns from running dry and to prevent mixing of each new aliquot with the previous aliquot. In this invention, the liquid flow of the column stops when the meniscus of the liquid above the column bed reaches the top frit of the column. In some embodiments, there is no top frit and the flow of liquid stops when it reaches the top of the bed of medium. The timing for addition of the next aliquot is based on the liquid reaching the top frit (or top of the bed) on the slowest running column. No column runs dry because the flow of the liquid through the column pauses when the liquid reaches the top of the column As a consequence, the new aliquot does not mix with residual from the previous aliquot in any of the columns. The various aliquots of liquid (conditioning solvent, sample, eluent or other solvents) are added and a preparative liquid chromatography separation is performed with a single column or across a plate or rack of columns. This method is effective in spite of varying backpressures and flow rates of the various columns found from column-to-column or within the plate or rack. The invention can be performed with an automated robotic handler or semi-automated robotic liquid handler. The invention can be applied to any aqueous type chromatographic methods including gel filtration, buffer exchange, desalting, ion exclusion, ion exchange, affinity, reverse phase, aqueous normal phase, hydrophobic interaction, hydrophilic interaction and any type of aqueous-based or partially aqueous-based chromatographic system as described below.

### BRIEF DESCRIPTION OF THE FIGURES

FIG. 1 depicts an embodiment of the invention where the chromatography column body is constructed from a tapered pipette tip.

FIG. 2 is an enlarged view of the chromatography column of FIG. 1.

FIG. 3 depicts an embodiment of the invention where the gravity column is constructed from two cylindrical members.

FIGS. 4 and 5 show the packing of a gravity chromatography column.

FIG. 6 depicts an example of a gel filtration desalting columns with a collection plate and transfer tips.

FIG. 7A depicts a top view of a rack or plate for holding the columns. FIG. 7B depicts a cut-away view of a rack or plate.

FIG. 8 depicts an addition of a sample aliquot and chaser elution aliquot to a gravity chromatography column.

FIGS. 9-13 show successive stages in the construction of gravity chromatography column.

FIG. 14 depicts the deck layout for a PhyNexus, Inc. MEA robotic liquid handler instrument.

FIG. 15 depicts the deck layout for a Beckman Biomek robotic liquid handler system.

### DETAILED DESCRIPTION OF THE INVENTION

This invention provides an automated or semi-automated, multiplexed, preparative gravity column liquid chromatography apparatus, columns and process. The columns may be operated manually. The gravity columns are small in diameter and can be operated with a 96-well or 384-well format. For the 96-well format, 1-96 columns may be operated in parallel. For the 384-well format, 1-384 columns can be operated in parallel.

In some embodiments, the columns are arranged in a rack. This arrangement is called the rack format. In other embodiments, the columns are integrated into the wells of a deep-well plate, which is designated the plate format. The 96-well rack or plate format consists of columns with 8 rows and 12 columns with 9.0 mm center-to-center spacing. That is, when columns arranged in the 96-well format are viewed from above, the distance between the centers of two adjacent columns will be 9.0 mm. The 384-well rack or plate format consists of columns with 16 rows and 24 columns with 4.5 mm center-to-center spacing.

In order to fit the chromatography columns into a 96-well format or 384-well format, the diameter and cross sectional area of the columns must be limited. This limits the volume of the liquid aliquot that can be applied to the top of the columns. Thus, the columns of the invention have a relatively small bed volume and cross sectional area.

Chromatography is a process where columns containing chromatographic media are used in one directional eluent flow. In a vertical, gravity column, the eluent flow is from the top of the column to the bottom of the column. Columns are conditioned with a conditioning solvent and then an injection of a sample is made to the top of the column. The sample is separated into various species using a developing eluent flow initiating at the top of the column and exiting the bottom. Sample materials are separated from each other with a partitioning process of the various components between the mobile and stationary phases. Separations of sample components depend on the relative affinity of the materials for the two phases. Components that have a high affinity for the stationary phase or the chromatographic media are retained on the column longer than materials that have a lower affinity for the stationary phase and partition more into the mobile phase.

Parameters that are considered in the addition of liquid aliquot to the head of a chromatography column include sample type and matrix buffer, elution solvent, column dead volumes, packing uniformity, sample injection volumes, band spreading, peak collection, total volume collection, aliquot mixing, and other parameters. These considerations make the addition of liquid aliquots to the top of the gravity columns while preserving the separation very difficult, especially as the columns become smaller and the size of the aliquots becomes smaller.

In certain embodiments, these processes are performed by liquid handlers. Because the columns have very small bed volumes and small cross sectional areas only very small aliquots of liquid and/or mass amounts of material can be applied to the columns without overloading the column capacity. However, small aliquots of liquid can exert only a small gravity force on the head of the column bed. There may not be sufficient force to push the liquid through the column bed. Capillary action of liquid to the wall of the columns or to the spaces between the column beads may present a counter force to gravity flow and may prevent liquid flow through the column. Too high of column backpressure may prevent liquid flow through the column. Since the chromatographic columns can fit into a 9.0 mm or 4.5 mm center-to-center format, the diameter of the chromatographic column is limited. For columns having the same length, smaller diameter columns will have higher backpressures than larger diameter columns. The low cross-sectional areas and small liquid aliquots used with these columns exhibit high resistance to liquid flow compared to the forces produced by the gravity of the small aliquots of liquid placed at the head of the columns. Yet the columns of this invention allow liquid aliquots of sample, eluents, buffers and solvents are able to flow through the columns under gravity conditions. Furthermore, very small aliquots of liquids ranging from 2-20 μL 5-100 μL and 10-200 μL and 10-1000 μL can be applied to the head of the column. In other words, small aliquots of 2, 5, 10, and 20 up to 100 μL and larger produce enough gravity force to allow the liquid to flow into columns of the invention. Aliquots larger than about 1000 uL can be added if a longer column body in the rack or plate is used or if an adapter is held above the rack above the rack or plate. Thus aliquots of 2-2000 uL and 2-5000 uL can be added to the head of the columns of the invention.

Collection of small volumes of purified material is also necessary. It is important to accurately and precisely collect the liquid volume of interest, not only for one column but for an entire column set being run in parallel in which the collection is performed simultaneously. But this is a problem that cannot be solved without employing new technology. Another problem to be solved is the prevention of air entering the column. Air entering the column will cause the liquid flow through the column to channel resulting in non uniform interaction of the stationary and liquid phases. This will change the flow characteristics of the column and will also harm the separation.

These problems are solved in part by using paused flow chromatography. The term, “paused flow chromatography” as used herein, is defined as a process in which the flow stops before the next aliquot of liquid is added. In this manner, mixing of the liquid aliquot with the previous liquid aliquot is avoided. This is accomplished in parallel, 1-96 at a time or 1-384 at a time. Interestingly, the time of the paused flow will vary from column to column because each column will have a different flow rate. Surprisingly, separations can still be performed in parallel. The paused flow operation can be performed many times within the chromatography separation process, normally with each aliquot addition. All of these operations are counterintuitive because conventional chromatography wisdom and theory teaches otherwise. Conventional chromatography teaching states that diffusion will result from paused flow and will destroy the separation to some degree. Furthermore since each column behaves differently i.e. the flow rate through the column is different, any negative impact to the separation will vary from column to column. Also, pausing the flow at different times for different columns could negatively affect separations from run to run and separations run in parallel. In effect, each column separation would be different from the next so there would be no motivation to develop a pause flow system for small columns because separations would be reproducible or useful.

The columns used in the apparatus have been designed and manufactured to have similar backpressures and flow rates. There are no air gaps between the frit and top of the column bed that may cause a disruption of flow. But the column bed compression is controlled to allow gravity flow for the small columns.

The various aliquots of liquid (conditioning solvent, sample, elution solvent or other solvents) are added without any column running dry. A paused flow system of chromatography is used. In this method of the invention, the liquid flow through the column stops when the meniscus of the liquid above the column bed reaches the top frit of the column. Surprisingly, when the liquid reaches the top of the column bed, the force of gravity forcing the liquid is matched by air from being prevented to flow into the column and the flow pauses. In some embodiments, no top frit is present and the liquid stops flowing when it reaches the top of the bed of medium although these columns are more difficult to design and produce. In some embodiments, the flow stops when the liquid reaches the top frit of the column. Surprisingly, the flow stops after the addition of small liquid aliquots. Surprisingly, air does not enter the column bed. Surprisingly, the flow restarts due to gravity when a new aliquot of liquid is added to the top of the column.

The timing for addition of the next aliquot is based on the liquid reaching the top frit for the slowest running column of two or more columns within the plate or rack. The invention can be applied to any aqueous type chromatographic methods including gel filtration, buffer exchange, desalting, ion exclusion, ion exchange, affinity, reverse phase, aqueous normal phase, hydrophobic interaction, hydrophilic interaction and any type of aqueous-based or partially aqueous-based chromatographic system provided the following criteria are fulfilled.

The subject invention involves methods and devices for separating or treating molecules from a sample solution using a packed bed of chromatographic medium. The media can be water-swollen gel-type gel filtration beads, silica gel, ion exchange, hydrophilic materials, hydrophobic materials, reverse phase or other types of beads. The methods, devices and reagents of the invention will be of particular interest to the life scientist, since they provide a powerful technology for treating biomolecules and other molecules of interest. However, the methods, devices and reagents are not limited to use in the biological sciences, and can find wide application in a variety of preparative and analytical contexts. The columns of this invention are used for aqueous-based elution systems of chromatography including size exclusion chromatography, gel filtration chromatography, buffer exchange, and desalting sample preparation and aqueous normal phase chromatography and other types of chromatography. The columns of this invention also are used in organic solvent and aqueous-based elution systems used in other types of chromatography including chaotropic normal phase chromatography and some types of reverse phase chromatography.

The invention provides separation columns many of which are characterized by the use of relatively small beds of chromatography media with small cross sectional areas, and are used with small volumes of solvents and buffers under gravity flow. The columns of the invention have or employ different properties in order to improve and automate performance of gravity flow chromatography manually or with semi-automated and liquid handler robotic systems.

In order to perform chromatography on an automated or semi-automated system the steps of liquid aliquot addition and collection must be automated. Column conditioning can be done manually if desired. The conditioning step may be performed immediately before using the column or the conditioning step may be done several days or weeks before the columns are used. Conditioning the column involves removing the glycerol and replacing the interstitial liquid and occluded liquid inside the beads with water or buffer. Glycerol is used to keep the resin swollen but must be removed before use for desalting or gel filtration separations. Once the glycerol is removed the columns must be kept wet with constant contact with water or buffer.

The gravity column separation steps can be manual, automated or semi-automated. The liquid flow through the column starts from the top of the column and the liquid exits at the bottom of the column. The gravity of the liquid on top of the column bed is the force used for passing liquid through the column. As in any chromatographic system, different liquid solutions are forced through the column including conditioning solvents or buffers, the sample, the chaser or eluent volume or volumes. The sample component of interest (the purified material) is collected at the appropriate time when the volume fraction containing the material of interest exits the bottom of the column. The collection is performed after a pause in flow when a new aliquot of liquid is added. Generally, the amount of liquid collected is the same as the aliquot of liquid that is added to the top of the column. Collection of the purified material is performed with a process that allows the collection of very small volumes of liquid at precise elution volumes within the chromatography separation process. This collection process can be performed in a parallel manner allowing precise collection of materials across an entire rack or plate if desired. In some embodiments, the process can be performed manually with single columns or a few columns run in parallel.

After conditioning, the first step in a separation process is the addition of the sample. The injection of the sample and the addition of all other liquid aliquots is performed by adding the appropriate liquid to the top of the column in a multiplexed manner with a pipetting system. In some embodiments, the aliquots are added with a liquid handler. In some embodiments the aliquot is added with a pipette. The liquid is allowed to flow down to the top frit and the flow stops. The liquid aliquot containing the sample is introduced to the top of the column without introducing air to the column bed. The liquid aliquot is added so that it is in direct contact with the top frit and no air bubbles are present that will prevent frit contact with the aliquot. The sample is allowed to pass through the column by gravity flow until the flow stops. The size of the injection will affect the performance of the column. Smaller injection aliquots may provide the best resolution of the samples species being separated on the column. In some embodiments, the size of the injection aliquot will range from 10 uL to the bed size of the column being used.

Most of the initial liquid from the sample is drained to a waste collection plate, but at the appropriate time in the chromatographic process, the rack or plate of columns is positioned over a collection plate. Then, an aliquot of a second liquid is added and the drop or drops containing the component of interest from each column in the rack or plate are collected. The second liquid can be an elution solvent. The rack or plate is moved at the appropriate time to collect the component of interest. The bottom of the columns may touch the sides of the wells of the collection plate so that any drop that exits the column is collected in the collection plate. This process may be repeated one or more times chromatographic separation process if more than one component of interest in the sample is being separated and collected. In some embodiments, all of these steps are performed in an automated fashion using a liquid handling robot. In certain embodiments, isocratic or gradient elution processes may be used.

In those embodiments that utilize a liquid handler, the timing for addition of aliquots can be determined empirically based on the slowest flowing column. The time period between the addition of new aliquots is at the same time or longer than the time needed for the flow of the slowest flowing column to pause. The timing is chosen such that the previous aliquot has reached the top frit or top of the column bed and the flow has stopped. Once the timing is determined for the addition of aliquots, the same timing can be used for subsequent separations.

Gravity liquid chromatographic columns operate under gravity flow of liquid with the pressure provided by the force of the liquid above the head of the column. Packed bed columns inherently have back pressures that vary from column-to-column. These two factors lead to flow rates that vary between columns within the plate or rack. In an automated system with of the columns of the invention being operated in parallel, the addition of aliquots to the columns is performed at the same time for all columns. The addition of the next aliquot is performed according to timing dictated by a computer program used by the liquid handler. For optimum column performance in gravity column liquid, each aliquot of liquid added to the top of the column should be added at just the right time. It is desirable to minimize mixing of any liquid from the previous aliquot remaining above the column bed with the new aliquot of liquid, but if too much time elapses, the column could run dry and air could be introduced into the column bed. Aliquots must be added before any one column of the rack or plate runs dry but where there is still some liquid above the column bed or frits. The meniscus of liquid on top of the columns will vary from column to column and the timing of the aliquot addition of the next volume is timed to minimize the amounts of liquid at the heads of the columns. The pressure of the liquid is dependent on the cross sectional area of the column and the volume of liquid above the frit or bed of medium.

It is surprising that there would be enough pressure for flow to reach near the top of the columns for these small columns because the gravity pressure of the liquid above the small cross sectional areas that must be used when the columns are in a 96 well format or a 384 well format. Indeed, if the columns are not packed correctly, the back pressure is too high and there is not enough pressure. Also, as the diameter of the gravity column is decreased, capillary action of the liquid moving up the column is a force that counteracts gravity flow. Capillary action works against the gravity flow due to head pressure. Capillary action will increase as the column diameter decreases.

Since all columns flow at slightly different flow rates, it is surprising that this gravity flow operation can be performed with automated, timed steps controlled by a computer program and still be able to get useable separations with the columns. This embodiment can be applied to any aqueous type chromatographic method including gel filtration, buffer exchange, desalting, ion exclusion, ion exchange, affinity, reverse phase, aqueous normal phase, hydrophobic interaction, hydrophilic interaction and any time of aqueous-based or partially aqueous-based chromatographic system.

In the paused flow system of chromatography of the invention, the liquid flow of the column stops when the liquid reaches the frit or top of the column bed. The timing for addition of the next aliquot is based on the time the liquid reaches the top frit (or top of the bed) of the slowest running column, two or more columns, or of the entire plate or rack. This system can be applied to any aqueous type chromatographic methods including gel filtration, buffer exchange, desalting, ion exclusion, ion exchange, affinity, reverse phase, aqueous normal phase, hydrophobic interaction, hydrophilic interaction and any time of aqueous-based or partially aqueous-based chromatographic system provided the following criteria are fulfilled.

1. The solvent must have the properties to be able to interact with the frit pores causing liquids to function in a paused flow manner. The bonding must be of a type that allows, under gravity flow conditions, the flow of liquid into and through the column and does not permit the passage of air through the column. When no top frit is present, the flow of liquid must stop before all the liquid enters the bed. Aqueous solvents can be used in a paused flow manner. Aqueous solvents that contain organic solvents can also be used in a paused flow manner. Organic solvents such as alcohols, ethanol, 2-propanol, acetone, acetonitrile and others can additionally be used in a paused flow manner. Water-miscible liquids such as alcohols, propanol, ethanol, methanol, aprotic solvents can be used or any nonpolar solvent can be used as long as the flow of the liquid stops at the top of the column and air does not enter the column.
2. The gravity flow must have sufficient pressure to force the flow of liquid to reach the top frit or top of the column bed. The pressure of the liquid is dependent on the cross sectional area of the bed and the volume of liquid above the bed. It is surprising that there is enough pressure for the liquid to reach the top frit of the column (or the top of the bed) because of the small cross sectional areas that must be used when the columns are arranged in a 96-well format or a 384-well format. Sometimes the aliquot applied to the top of the column can be very small, sometimes as small as 2 uL, and yet enough force is produced for the aliquot to flow into the column bed.
3. The column packing material must be of a type and size and packed in a way that permits the use of gravity flow to force liquids through the column.
4. The column dimensions must be of a type and size that permits the use of gravity flow to force liquids through the column.
5. The columns must perform with sufficiently similar flows such that the flow process can be done in parallel and under timed conditions.

In the columns and method of the invention, the gravity flow will stop for each individual column as the liquid reaches the top frit or top of the column bed. The meniscus of the liquid will flow to the top of each column individually and the flow will stop at the frit of each column. In some cases, the flow will stop at the top of the column bed without a top frit. The next round of aliquots of liquid are added when the meniscus of the liquid of the slowest flowing column reaches the frit. In this manner, mixing of previous solution in the column with the new aliquots of liquid is minimized even when multiple columns are used in parallel in an automated apparatus.

It is surprising that liquid flow stops at the frit or top of the column bed. In fact, it is surprising that there is sufficient liquid flow using these small columns that possess very low head pressures. It is surprising that this operation can be done in parallel. It is surprising that this paused flow operation can be performed with automated, timed steps controlled by a computer program. Employing this type of aliquot addition to many columns in a rack or plate will result in a paused flow process that will vary from column-to-column. Paused flow in liquid chromatography is not desirable. Conventional wisdom teaches that paused flow will harm the chromatography separation process due to component bands spreading as a result of longitudinal diffusion along the column. In addition, the band spreading can vary from column-to-column if the paused flow occurs at different times for each column. Surprisingly, good column separation performance can be achieved with paused flow elution methods of the invention.

Collection of the material of interest must be done in an accurate and precise manner. Under normal operation, conditioning of the column, sample loading or injection, washing or developing the column is performed with the solvent flow to waste. The waste container or containers collect the liquid from the various steps. Prior to collection, it is helpful for the drop hanging from each of the columns to be consistent. In some embodiments, the wash liquid touches the bottoms of the columns or the columns are moved to touch a surface or blot the end of the column or columns. This is done so as the column or rack or plates of columns are lifted, the drop is consistent form column to column. The column or rack or plate of columns is moved to the collection plate. In some embodiments, the ends of the columns touch the wall or bottom of the wells in vials or the collection plate with capillary action drawing the liquid existing above the column bed to be drawn through the column and into the vial or well. In manual operation, the column may be held in a holder or simply be inserted into a vial or plate so the bottom of the column naturally is in contact with the well of the vial or plate. When the final collection chaser or elution is added to the top of the column, the material of interested is collected in the wells of the collection plate. Adapters may be positioned in one or more of these operations to position the columns at the most advantageous distance above the collection plate. The volume of liquid collected may be the same or similar to the volume of aliquot of the elution solvent added to the column in the elution step.

The volume of purified material can be expressed as a percentage of the column bed volume. In some embodiments, the volume of purified material is in the range of 2% to 200%, 2% to 100% or 5% to 100% of the bed volume. In other embodiments, the volume of purified material is greater than 200% of the bed volume. In certain embodiments, the volume of purified material can be expressed in absolute terms. In some embodiments, the volume can be in the range of 5 μL to 600 μL or 20 μL to 90 μL. In some embodiments, the volume of purified material obtained from the column has a coefficient of variation of less than 20. In certain embodiments, the volume of purified material obtained from the column has a coefficient of variation of less than 10.

### DEFINITIONS

In describing and claiming the present invention, the following terminology will be used in accordance with the definitions set out below.

The term “gravity liquid chromatography” is a separation process by which components are separated on a bed of stationary phase. A liquid mobile phase is used to develop the separation and elute the material of interest. Gravity forces the liquid through the column.

The term “semi-automated” for gravity liquid chromatography process is defined as a process where the liquid aliquot is added to one or more columns at the same time. Semi automated may mean that only part of the process is automated and part of the process is manual.

The term “automated” for gravity liquid chromatography process is defined as a process where the liquid aliquot is added to one or more columns at the same time and the various liquid aliquots are added according to a timed computer program.

The term “manual” for gravity liquid chromatography process is defined as a process where the liquid is added manually to one or more columns and the various liquid aliquots are added according to visually determining when the flow has paused or the liquid meniscus has reached the top of the column.

The term “meniscus” is the top portion of the liquid aliquot that has been added to the top of the gravity flow column.

The term “bed channel or channeling” is the inconsistent flow path of liquid through a column.

The terms “pause” and “stop” are used interchangeably with reference to flow through the column and refer to the phenomenon of the flow of the liquid stopping when sample, washes and elution liquids are added to the top of the column and the flow pauses or stops when the liquid reaches the top of the column bed. The flow restarts when another liquid aliquot is added to the top of the column bed.

The term “aliquot mixing” is the mixing of the new aliquot of liquid to the aliquot of liquid that was previously added to the top of the column.

The term “paused flow” is the automatic stopping of liquid at the top frit of a column or the top of the column bed. Liquid flows to the top of the bed, but air does not enter the bed and prevent flow of the next liquid aliquot.

The term “plate or rack row and column” is the rows and columns of a 96-well format or a 384-well format. For a 96-well rack or plate format there are 8 rows and 12 columns with 9.0 mm center-to-center spacing. For a 384-well rack or plate format there are 16 rows and 24 columns with 4.5 mm center-to-center spacing.

The term “plate or rack of column or columns” refers to the column or columns that are packed and placed into a rack or plate or to the column or columns that are packed into a plate. The terms are used interchangeably. Rack may contain columns fitted or assembled into a fixture of 1-96 columns or 1-384 columns. Columns may be used individually or used in a rack. A plate of columns may be a fully molded assembly where 96 columns or 384 columns are packed.

The term “column cross sectional area” refers to the area of the top of the column presented to the liquid aliquots added to the column. The column cross sectional area shape can be round, square or any shape. The column dimension may fit into 9.0 mm or 4.5 mm center to center spacing for automated operation or into plates or vials for manual operation.

The term “cross-sectional area” refers to the area of a cross section of the frit at the head of the column or the bed of chromatography media, i.e., a planar section of the bed generally perpendicular to the flow of solution through the bed and parallel to the frits. In the case of a cylindrical or frustoconical bed, the cross section is generally circular and the cross sectional area is simply the area of the circle, area=pi×r². For a square or rectangular shaped bed, area=l×d. The average cross-sectional area of the frit can be quite small in some of the columns of the invention. Examples include cross-sectional areas of less than about 100 mm², less than about 81 mm² about 64 mm², less than about 5.1 mm², or less than about 4 mm². Thus, some embodiments of the invention involve ranges of cross sectional areas extending from a lower limit of 4, 10, 12, 15 or 20 mm² to an upper limit of 30, 40, 50, 60, 70, 80, 90 or 100 mm².

The term “bed volume” as used herein is defined as the volume of a bed of chromatography media in a chromatography column. Depending on how densely the bed is packed, the volume of the chromatography media in the column bed is typically about one third to two thirds of the total bed volume; well packed beds have less space between the beads and hence generally have more beads packed into the column and lower interstitial volumes.

The term “exclusion volume” of the bed refers to the volume of the bed between the beads of chromatography media that is accessible to one of the solvents or buffers used in the gel filtration columns, e.g., aqueous sample solutions, wash, conditioning, and chaser solutions and elution solvents. For example, in the case where the chromatography media is a chromatography bead (e.g., agarose or sepharose), the exclusion volume of the bed constitutes the solvent accessible volume between the beads, but excluded from the solvent accessible internal regions of the bead, e.g., solvent accessible pores.

The terms “analyte”, “analytes”, “material”, “materials”, “component” and “components” are used interchangeably as used herein. The terms refer to molecule or molecules of interest in a sample. They include biomolecules and other molecules of interest in a sample.

The terms, “eluent”, “wash”, “chaser”, “buffers” and “solvents” are used interchangeably herein.

The term “elution liquid” refers to buffer or solvent that is used to wash or elute material from the gravity column.

The term, “dead volume” as used herein with respect to a column is defined as the interstitial volume of the chromatography bed, tubes, membrane or frits, and passageways in a column. Some preferred embodiments of the invention involve the use of low dead volume columns, as described in more detail in U.S. Pat. No. 7,482,169.

The term, “elution volume” as used herein is defined as the volume of elution liquid added to the top of the column and into which the analytes or materials are eluted and collected. The terms “elution liquid” and “chaser” liquid aliquot and the like are used interchangeably herein.

The terms, “gel filtration column” and “gel filtration tip” and “rack of gel filtration columns” and “plate of gel filtration columns” as used herein are defined as a column device used in gravity flow used in combination with robotic liquid handler containing a bed of solid phase gel filtration material, i.e., gel filtration media.

The term, “chromatography gravity columns” and “gravity chromatography columns” refer to columns of the invention in which the force of gravity is used to force the sample, buffers, eluents and solvents through the columns.

The term, “frit” as used herein is defined as porous material for holding the gel filtration media in place in a column. A chromatography media chamber is typically defined by a top and bottom frit positioned in a chromatography column. The top frit allows liquid to enter and pass into the through the column under gravity flow, but does not allow air to enter the column under gravity flow. In some embodiments of the invention, the frit is a thin, low pore volume fabric, e.g., a membrane screen. In some embodiments of the invention, the frit is a porous or sintered material. In some embodiments, the top frit is absent and chromatography media positioned above the bottom frit allows liquid to enter and pass through the column under gravity flow, but does not allow air to enter the column under gravity flow conditions.

The term, “lower column body” as used herein is defined as the column bed and bottom membrane screen of a column.

The term, “membrane screen” as used herein is defined as a woven or non-woven fabric or screen for holding the column packing in place in the column bed, the membranes having a low dead volume. The membranes are of sufficient strength to withstand packing and use of the column bed and of sufficient porosity to allow passage of liquids through the column bed. The membrane is thin enough so that it can be sealed around the perimeter or circumference of the membrane screen so that the liquids flow through the screen.

The term, “sample volume”, as used herein is defined as the volume of the liquid of the original sample solution from which the analytes are separated or purified.

The term, “upper column body”, as used herein is defined as the chamber and top frit or membrane screen of a column.

The term, “biomolecule” as used herein refers to biomolecule derived from a biological system. The term includes biological macromolecules, such as a proteins, peptides, polysaccharides, and nucleic acids.

The term, “protein chip” is defined as a small plate or surface upon which an array of separated, discrete protein samples are to be deposited or have been deposited. These protein samples are typically small and are sometimes referred to as “dots.” In general, a chip bearing an array of discrete proteins is designed to be contacted with a sample having one or more biomolecules which may or may not have the capability of binding to the surface of one or more of the dots, and the occurrence or absence of such binding on each dot is subsequently determined. A reference that describes the general types and functions of protein chips is Gavin MacBeath, Nature Genetics Supplement, 32:526 (2002).

Different types of chromatography will require different types of conditioning and elution solvents. Some solvents and buffers are aqueous based and are useful in gel filtration, ion exchange, normal phase chromatography and other types of chromatography. Other solvents are mixtures of aqueous solvents and organic solvents and are useful in reverse phase, ion exchange, normal phase, and other types of chromatography. Experiments were performed in 100% buffers, mixtures of aqueous and organic solvents and 100% organic solvents. Columns of the invention were found to have properties that allowed the use of paused flow chromatography.

In some embodiments, the instant invention provides one or more chromatographic columns in a rack or plate format with the packed bed column comprising: a column body having an open upper end, an open lower end, and an open channel between the upper and lower end of the column body; a bottom frit bonded to and extending across the open channel; a top frit bonded to and extending across the open channel between the bottom frit and the open upper end of the column body, the top frit having a low pore volume, wherein the top frit, bottom frit, and column body define an chromatography media chamber; and a bed of chromatography media positioned inside the chromatography media chamber, said bed of chromatography media having a volume of less than about 4000 μL.

Due to natural variation, packed bed columns naturally have different densities even if packed with the same packing material. The column backpressures will vary column-to-column. Therefore the flow rate of a given volume of liquid through the columns will vary column-to-column. Also, the flow rate will vary as a given aliquot of liquid decreases in volume as liquid flows through a particular column thereby further exacerbating the column-to-column variation. The flow variation is even greater for the columns of the invention since the gravity pressure forcing liquid flow through the columns is a very low. Very small aliquots of liquid of 2-100 uL and 5-1000 uL have very low gravity pressures. In some embodiments of the columns and flow conditions of the invention, the flow variation from column-to-column is no greater than 50% or is no greater than 25% relative of the fastest flowing column to the slowest flowing column with these liquid aliquots.

In order to obtain maximum separation performance, the addition of new aliquots to the column bed should be executed exactly at the time when the liquid meniscus just reaches the top of the column bed. In a manual gravity flow column, the timing of this operation is usually determined using visual feedback. The aliquot of liquid is usually added just as the liquid reaches the top of the column bed. Allowing the liquid to flow past the top of column bed will introduce air into the column bed which may degrade column performance. This degradation could manifest in changing the flow rate through the column, peak spreading, channeling or other harmful chromatographic behavior. In some embodiments of the invention, the addition of aliquots is performed before any one column of the rack or plate has liquid flowing past the top of the column bed such that air does not enter the column. That is, the top frit or top of the bed of medium should not become dry.

Small column volumes and small solvent volumes also make collection of the material of interest more difficult. The collection of volumes of liquid 2-500 uL, 2-100 uL, 2-50 uL, 2-40 uL, 2-30 uL, 2-20 uL, 2-10 uL, and 5-10 uL can be performed. In some embodiments, the volume of the aliquot of liquid intended or chosen to be collected is the same volume or a similar volume that was added to the top of the column. The chromatography of the column or columns has been developed to the stage an aliquot is to be collected. The column is operated in a paused flow form and with the liquid meniscus at the frit of the column. The column, columns, plate or rack of columns is moved to a collection plate or vials. An aliquot of liquid is added to the columns and the volume flows through the column. The drop that forms at the end of the column is collected by touching the drop to the collection plate or vial to drain the volume into the plate or vial.

In some embodiments, the flow through the column is performed in a paused flow manner. The flow through the column is not continuous and only flows when there is a force of a liquid segment above the column frit. Flow occurs only when liquid is above the head of the column. Flow stops when the meniscus of liquid reaches the top frit or the top of the column bed.

In some embodiments, fractions of liquid are collected below in a collection well, wells or plate.

In some embodiments, the columns are contained in a rack or plate that can move from position to position with a robotic arm.

In some embodiments, the bed of extraction media comprises a packed bed of resin beads. Non-limiting examples of resin beads include water swollen gel resins and resins with hydrophilic surfaces.

In certain embodiments of the invention, the column comprises a packed bed of resin beads. Non-limiting examples include agarose- or sepharose-based resins, cellulose, polyacrylamide, dextran, silica, functionalized silica, silica gel and other polymer materials.

In certain embodiments of the invention, the bed of chromatography media has a volume of between about 5 μL and 4000 μL, between about 100 μL and 2000 μL, or between about 200 μL and 1000 μL.

In certain embodiments of the invention, the bottom frit and/or the top frit is/are less than 3 mm, less than 2 mm thick, less than, 1 mm thick, less than 500 microns thick, less than 200 microns thick and less than 100 microns thick.

In certain embodiments of the invention, the bottom frit and/or the top frit has/have a pore volume of 20, 10, 5, 1 μL or less.

In certain embodiments of the invention, the bottom frit and/or the top frit is a porous sinter, fabric, screen or membrane comprised of nylon, PEEK, PVC, polyester, polypropylene, polyethylene, polyolefinic, glass, steel, metal or ceramic frit.

In certain embodiments of the invention, the column body comprises a PVC, delrin, nylon, polyolefinic, polycarbonate, polypropylene, polyethylene, metal, or ceramic material.

In certain embodiments of the invention the column is configured into a plate or rack of columns with suitable 9.0 mm center-to-center column configuration to be used in a robotic liquid handler.

In certain embodiments of the invention the column is configured into a plate or rack of columns with suitable 4.5 mm center-to-center column configuration to be used in a robotic liquid handler.

In certain embodiments of the invention, the column body comprises a plate, luer adapter, syringe, cylinder, tube or pipette tip.

In certain embodiments of the invention, the column comprises a lower tubular member comprising: the lower end of the column body, a first engaging end, and a lower open channel between the lower end of the column body and the first engaging end; and an upper tubular member comprising the upper end of the column body, a second engaging end, and an upper open channel between the upper end of the column body and the second engaging end, the top membrane screen of the chromatography column bonded to and extending across the upper open channel at the second engaging end; wherein the first engaging end engages the second engaging end to form a sealing engagement. In some of these embodiments, the first engaging end has an inner diameter that matches the external diameter of the second engaging end, and wherein the first engaging end receives the second engaging end in a telescoping relation. The first engaging end optionally has a tapered bore that matches a tapered external surface of the second engaging end.

In certain embodiments of the invention, a gravity chromatography column adaptor is used to position the plate or rack of columns above the waste collection plate or vials and/or the elution collection plate or vials.

The invention further provides a method for separating a material or materials from a sample solution comprising the steps of introducing a sample solution containing a material or materials into the packed bed of chromatographic media packed into the bed of the column of the invention wherein the chromatographic media has affinity for one or more components in the sample, introducing a solvent or a series of solvents into the bed of chromatographic media, whereby at least some fraction of a material or materials are eluted from the column or columns and collected into a capture well, plate or rack of vials.

The invention further provides a method for separating a material or materials from a sample solution comprising the steps of introducing a sample solution containing a material or materials into the packed bed of chromatographic media packed into the bed of the column of the invention wherein the chromatographic media has affinity for one or more components in the sample, introducing a solvent or series of solvents into the bed of chromatographic media in paused flow mechanism whereby the addition of the next aliquot of liquid is added after the meniscus of the liquid above the column has reach the frit of the slowest flowing column, whereby at least some fraction of a material or materials are eluted from the column or columns and collected into a capture well, plate or rack of vials. The chromatographic methods of the invention include aqueous based elution systems of chromatography including size exclusion chromatography, gel filtration chromatography, buffer exchange, and desalting sample preparation and aqueous normal phase chromatography and other types of chromatography. For the purpose of this invention, size exclusion chromatography, gel filtration chromatography, desalting and buffer exchange are considered to be equivalent. The chromatographic method of the invention also include organic solvent and aqueous based elution systems used in other types of chromatography including chaotropic normal phase chromatography and some types of reverse phase chromatography.

The invention further provides a method for separating a material or materials from a sample solution comprising the steps of introducing a sample solution containing a material or materials into the packed bed of chromatographic media packed into the bed of the invention, wherein the chromatographic media comprises a water swollen or buffer swollen matrix having pores either larger or smaller than the material or analyte, whereby the analyte either enters the pores or is excluded from the pores of the gel filtration media; introducing a chaser or eluent solvent into the bed of gel filtration media, whereby at least some fraction of the analyte is eluted from the gel filtration media and collected into a capture well, plate or rack of vials.

The invention further provides a method for separating an analyte from a sample solution comprising the steps of introducing a sample solution containing an analyte into the packed bed of gel filtration media of a desalting column of the invention, wherein the gel filtration media comprises an water swollen or buffer swollen matrix having pores larger than the analyte, whereby the analyte enters or partially enters the pores of the gel filtration media and other matrix material are excluded or partially excluded from the pores of the gel filtration media and discarded; introducing a chaser solvent aliquot or series of aliquots into the bed of gel filtration media, whereby at least some fraction of the analyte is eluted from the gel filtration media and collected into a capture well, plate or rack of vials and separated from other sample matrix components.

The invention further provides a method for separating an analyte from a sample solution comprising the steps of introducing a sample solution containing an analyte into the packed bed of gel filtration media of a desalting column of the invention, wherein the gel filtration media comprises an water swollen or buffer swollen matrix having pores smaller than analyte, whereby the analyte is excluded or partially excluded the pores of the gel filtration media and other matrix materials enter or partially enter the pores of the gel filtration media; introducing a chaser solvent aliquot or series of aliquots into the bed of gel filtration media, whereby at least some fraction of the analyte is eluted from the gel filtration media an collected into a capture well, plate or rack of vials and separated from the other sample matrix components.

The invention further provides a method for desalting or buffer exchanging an analyte from a sample solution comprising the steps of introducing a sample solution containing an analyte into the packed bed of gel filtration media of a desalting column of the invention, wherein the gel filtration media comprises an water swollen or buffer swollen matrix having pores smaller than analyte but large enough for buffer or salts to enter, whereby the analyte is excluded or partially excluded the pores of the gel filtration media and other matrix salts enter or partially enter the pores of the gel filtration media; introducing a chaser solvent aliquot or series of aliquots into the bed of gel filtration media, whereby at least some fraction of the analyte is eluted from the gel filtration media and collected into a capture well, plate or rack of vials and is desalted and/or contains a new buffer and is separated from the original sample matrix salt or buffer.

The invention further provides a method for affinity chromatography capturing and purifying a protein, nucleic acid or other biomolecule from a sample solution comprising the steps of introducing a sample solution containing an analyte into the packed bed of affinity media of a column of the invention, wherein the affinity media comprises an water swollen or buffer swollen matrix having affinity groups that capture biomolecules, whereby non specific materials are not retained and are washed away using a solvent or buffer, introducing a chaser solvent aliquot or series of aliquots into the bed of affinity media, whereby at least some fraction of the biomolecule is eluted from the affinity media and collected into a capture well, plate or rack of vials.

The invention further provides a method for ion exchange chromatography capturing and purifying a protein, nucleic acid or other biomolecule from a sample solution comprising the steps of introducing a sample solution containing an analyte into the packed bed of ion exchange media of a column of the invention, wherein the ion-change media contain groups that capture or exchange biomolecules, whereby non specific materials are not retained and are washed away using a solvent or buffer, introducing a chaser eluent solvent aliquot or series of aliquots into the bed of affinity media, whereby at least some fraction of the biomolecule is eluted from the ion exchange media and collected into a capture well, plate or rack of vials.

The invention further provides a method for normal phase chromatography capturing and purifying a nucleic acid or other biomolecule from a sample solution comprising the steps of introducing a sample solution containing an analyte into the packed bed of normal phase media of a column of the invention, wherein the normal phase media contain groups that capture or exchange biomolecules by interactions or chaotropic interactions, whereby non specific materials are not retained and are washed away using a solvent or buffer, introducing a chaser eluent solvent aliquot or series of aliquots into the bed of affinity media, whereby at least some fraction of the biomolecule is eluted from the normal phase media and collected into a capture well, plate or rack of vials.

The invention further provides a method for reverse phase chromatography capturing and purifying a protein, nucleic acid or other biomolecule from a sample solution comprising the steps of introducing a sample solution containing an analyte into the packed bed of reverse phase media of a column of the invention, wherein the reverse phase media contain groups that capture or exchange biomolecules or ion pairs of molecules, whereby non specific materials are not retained and are washed away using a solvent or buffer, introducing a chaser eluent solvent aliquot or series of aliquots into the bed of reverse phase media, whereby at least some fraction of the biomolecule is eluted from the affinity media and collected into a capture well, plate or rack of vials.

In certain embodiments, the invention provides a multiplexing of 2-96 columns in a 96-well format. The columns are of limited cross sectional area that can fit into a configuration of 9.0 mm center-to-center spacing. In other embodiments, the columns are arranged in a configuration of 4.5 mm center-to-center spacing in a multiplexing of 2-384 columns in a 384-well format. The columns may be any shape. For example, the horizontal cross section of the columns can be individual in a rack or in a plate and be circular, oval, square, rectangular or an irregular shape. In some embodiments, a plurality of columns is arranged in a 96 well format of 8 rows columns on one side and 12 rows of columns on the other side.

In some embodiments, a plurality of columns is arranged in a 384 well format of 16 rows of columns on one side and 24 rows of columns on the other side.

In certain embodiments of the method, the desalting column or columns are moved individually or in a rack into various stations in the robotic liquid handler.

In certain embodiments of the method, the desalting columns or rack or plates of column or columns are moved into various stations in the robotic liquid handler.

In certain embodiments of the method the side and/or bottom of the column or columns are in intimate contact with the waste and elution collection plate or vials below the columns.

In certain embodiments of the method drops of liquid exiting the column or columns come into intimate contact with the waste and elution collection plate or vials below the columns.

In certain embodiments of the method, aliquots of liquid are applied or deposited to the top of the column or columns with a pipette or liquid dispensing head in a liquid handler.

In certain embodiments of the method, the top frit has properties that allow liquid to flow through the frit and into the column, but the top frit does not allow air to flow into the column thereby stopping the flow of liquid until the next aliquot of liquid is added to the top of the column.

In some embodiments, this invention relates to methods and devices for separating, desalting or buffer exchanging an analyte from a sample solution using a gravity flow column. The column contains gel filtration media. The analytes can include biomolecules, particularly biological macromolecules such as proteins and peptides, polynucleotides, lipids and polysaccharides. The device and method of this invention are particularly useful in for proteomics sample preparation and analysis and for nucleic acid purification and analysis and other molecular separation and purification and analysis. The separation process generally results in the purification, desalting or buffer exchange of an analyte or analytes of interest.

In U.S. patent application Ser. No. 10/620,155, now U.S. Pat. No. 7,482,169, incorporated by reference herein in its entirety, methods and devices for performing low dead column extractions are described. The instant specification, inter alia, expands upon the concepts described in that application.

Gel filtration chromatography is a chromatographic method in which particles are separated based on their size or hydrodynamic volume. The method usually applied to large molecules such as proteins and other biomolecules such as polysaccharides and nucleic acids. Biologists and biochemists typically use a gel medium or packing material usually polyacrylamide, dextran or agarose.

The advantages of this method include good separation of large molecules from the small molecules with a minimal volume of eluent and that various buffers can be used with affecting the separation process all while preserving the biological activity of the analyte particles.

The underlying principle of gel filtration chromatography is that particles of different sizes will elute or travel through a stationary phase at different rates resulting in the separation of a solution of particles based on size. Provided that all analyte particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

Increasing the column length will enhance the resolution power of the column but will also increase column back pressure making gravity flow more difficult. Increasing the column diameter increases the capacity of the column but in this invention the diameter is limited by the configuration of the 96 well plate and rack. Proper column packing is important to maximize resolution: over-packed columns can collapse the pores in the beads, resulting in a loss of resolution and high and variable column backpressure. An under-packed column can improve the column backpressure but can reduce the relative surface area of the stationary phase accessible to smaller species, resulting in those species spending less time trapped in pores. Unlike affinity chromatography techniques, a solvent head at the top of the column can drastically diminish resolution as the sample diffuses prior to loading,

broadening the downstream elution. The void volume is the total space surrounding the gel particles in a packed column.

In gravity columns, the eluent is collected in volume aliquots known as fractions. In order to successfully operate the columns in parallel, the analytes or molecules of interest must travel down the column in parallel at more or less the same time.

The steps of using the columns are similar to the various types of separation chromatography. For example, for buffer exchange or desalting, the column is conditioned and the flow pauses. The sample is added with a new aliquot. The size of the sample is usually small so that it does not break through the end of the column and the flow pauses. Taking care that the drop at the end of the column is not large, the column is moved to a collection vial or plate. Then the desalted or buffer exchanged sample is eluted with an aliquot of chaser solvent or elution solvent and collected in the vial or plate. For other gel filtration applications, for example, size separations, further sequential aliquots of elution or chaser solvents may be added to collect fractions in sequential vials or plates with flow pausing for each collection.

For other types of chromatography, the procedure is similar. For example in affinity chromatography, after the column is conditioned, the flow pauses. The sample is added to the column. The volume of the sample in this case may be large in order to load up the column as much as possible. In some cases, excess sample may break through the column. After the sample is added, the flow pauses. The column may be washed to remove non specific bound material and the flow pauses. The first aliquot of elution solvent is added in order to start the elution process. The sample starts to elute at the head of the column but the aliquot of eluant is not large enough to elute material from the column and the flow pauses. Then the column is moved to a collection vial or plate taking care that the drop at the end of the column is not large. The column is positioned so that the end of the column touches the vial or plate. The next aliquot of elution liquid is chosen to elute and collect the bulk of the material from the column.

For ion pair, reverse phase chromatography, the column is conditioned and the flow pauses. The sample addition in this case may be smaller so retain the sharpness of the sample peak at the head of the column and the flow pauses. Several aliquots of elution liquid may be added to collect fractions with the flow pausing before each aliquot addition.

The design of the conditioning step, sample loading, washing, elution and collection volumes and flow pausing depends on the type of chromatography used and the separation desired. After column conditioning, an injection aliquot or addition of a small volume of sample is added to the column. The columns the desired material may be collected in with the next aliquot addition of elution solvent. Or the column may be washed with a wash solution and then the desired material may be collected next. Or the collection may be performed with the addition of a series of elution buffers or solvents.

For example the sample may be a complex mixture containing proteins of various sizes. To test how the columns perform as size exclusion chromatographic columns, the following fractionation of the sample may be performed.

1) add a small volume of buffer and collect the flow through. Repeat for 12 individual fractions. This can be extended indefinitely.
2) add a large volume and collect fractions over a discreet period of time.
3) add a volume of buffer and flow that to waste. This volume is large enough to reduce the number of fractions collected, but small enough to prevent the loss of the desired sample e.g. protein.
Similar steps can be done for other types of gravity flow chromatography including affinity, ion exchange, normal phase, ion pair reverse phase and other types of chromatography. For example, a step gradient of elution solvents can be added to the column with fractions collected for each solvent. Or multiple fractions can be collected with a single elution solvent. A liquid aliquot is added only the flow has paused. Liquid can be collected or discarded to waste. The full volume of a liquid aliquot or multiple fractions can be collected by moving column to an empty well as the buffer flows through the column.

Before describing the present invention in detail, it is to be understood that this invention is not limited to specific embodiments described herein. It is also to be understood that the terminology used herein for the purpose of describing particular embodiments is not intended to be limiting. As used in this specification and the appended claims, the singular forms “a”, “an” and “the” include plural referents unless the context clearly dictates otherwise. Thus, for example, reference to polymer bearing a protected carbonyl would include a polymer bearing two or more protected carbonyls, and the like.

Unless defined otherwise, all technical and scientific terms used herein have the same meaning as commonly understood by one of ordinary skill in the art to which the invention pertains. Although any methods and materials similar or equivalent to those described herein can be used in the practice or testing of the present invention, specific examples of appropriate materials and methods are described herein.

### Chromatography Columns

In accordance with the present invention there may be employed conventional chemistry, biological and analytical techniques within the skill of the art. Such techniques are explained fully in the literature. See, e.g. Chromatography, 5^{th} edition, PART A: FUNDAMENTALS AND TECHNIQUES, editor: E. Heftmann, Elsevier Science Publishing Company, New York (1992); ADVANCED CHROMATOGRAPHIC AND ELECTROMIGRATION METHODS IN BIOSCIENCES, editor: Z. Deyl, Elsevier Science BV, Amsterdam, The Netherlands, (1998); CHROMATOGRAPHY TODAY, Colin F. Poole and Salwa K. Poole, and Elsevier Science Publishing Company, New York, (1991).

In some embodiments of the subject invention, the packed bed of chromatographic media is contained in a column. Non-limiting examples of suitable columns are presented herein. It is to be understood that the subject invention is not to be construed as limited to the use of single chromatography bed columns, or in columns in general. For example, the invention is equally applicable to use with a packed bed of chromatography media as a component of a multi-well plate or rack.

### Column Body

The column body is a tube having two open ends connected by an open channel, sometimes referred to as a through passageway. The tube can be in any shape, including but not limited to cylindrical or frustoconical, and of any dimensions consistent with the function of the column as described herein. In some preferred embodiments of the invention the column body takes the form of a pipette tip, a syringe, a luer adapter or similar tubular bodies. In embodiments where the column body is a pipette tip, the pipette tip is modified to contain the chromatography media. The end of the tip wherein the bed of chromatography media is placed can take any of a number of geometries, e.g., it can be tapered or cylindrical. In some case a cylindrical channel of relatively constant radius can be preferable to a tapered tip, for a variety of reason, e.g., solution flows through the bed at a uniform rate, rather than varying as a function of a variable channel diameter. In some embodiments, one of the open ends of the column sometimes referred to herein as the open upper end of the column, is adapted for attachment to a pump head, either directly or indirectly for movement of the columns.

In some embodiments, column bodies are comprised of the wells within a deep-well plate. In these embodiments, the deep-well plate can be a 96-well or 384-well plate.

Columns may be located in a plate or rack. Column bodies can be of any size as long as they can be accommodated in a standard 96-well or 384-well format. In some embodiments, column bodies are made from 200 μL or 1 mL pipette tips.

The column body can be composed of any material that is sufficiently non-porous that it can retain fluid and that is compatible with the solutions, media, pumps and analytes used. A material should be employed that does not substantially react with substances it will contact during use of the chromatography column, e.g., the sample solutions, the analyte of interest, the chromatography media and conditioning and elution solvents. A wide range of suitable materials are available and known to one of skill in the art, and the choice is one of design. Various plastics make ideal column body materials, but other materials such as glass, ceramics or metals could be used in some embodiments of the invention. Some examples of preferred materials include polysulfone, polypropylene, polyethylene, polyethylene terephthalate, polyethersulfone, polytetrafluoroethylene, cellulose, cellulose acetate, cellulose acetate butyrate, acrylonitrile PVC copolymer, polystyrene, polystyrene/acrylonitrile copolymer, polyvinylidene fluoride, TEFLON and similar materials, glass, PEEK, metal, silica, and combinations of the above listed materials.

### Collection Plate Assembly

Single columns or a group of columns can be positioned into a rack of columns. The column bodies can be adapted into a plate format containing 96 or 384 columns or some fraction thereof. The rack or plate may be in the form of a gravity column holder or adaptor. The adaptor can be moved with robotic controllers and positioned above the waste collection plate or vials and the elution collection plate or vials. The collection assembly allows the drop coming off the end of the column to effectively be collected in the waste collection plate or vials and in the elution collection plate or vials. In some embodiments, the final drop coming off the end of the column touches the collection plate or vial so that the drop is collected.

### Chromatographic Media

The chromatography media used in the column is preferably a form of water-insoluble particle. Typically the analyte of interest is a protein, peptide or nucleic acid. The term “analyte” can refer to any material, sample component or compound of interest, e.g., to be analyzed, purified or simply removed from a solution.

Many of the chromatography media suitable for use in the invention are selected from a variety of classes of media. It has been found that many of these chromatography media and the associated chemistries are suited for use as solid phase gel filtration desalting, affinity, ion exchange, and other types of media in the devices and methods of this invention. Common gel resins include agarose, sepharose, polystyrene, polyacrylate, cellulose and other substrates. Gel resins can be non-porous or micro-porous beads. Soft gel resin beads, such as agarose and sepharose based beads, are found to work well in columns and methods of this invention. Other types of silica gel and polymer resin chromatography media work well in the columns and methods of the invention.

Use of the plate and rack format can limit the maximum bed volume of the column that can be used. For small columns, the aliquot must have enough gravitational force to force the liquid aliquots through the column. For the large columns, the configuration must allow 9.0 mm center to center formatting so that robotic liquid handlers and automation can be used.

The average particle diameters of beads of the invention are typically in the range of about 2 μm to several hundred microns, e.g., diameters in ranges having lower limits of 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 50 μm, 60 μm, 70 μm, 80 μm, 90 μm, 100 μm, 150 μm, 200 μm, 300 μm, 500 μm.

### Frits

In some embodiments of the invention, one or more frits is used to contain the bed of chromatography in, for example, a column. Frits can take a variety of forms, and can be constructed from a variety of materials, e.g., glass, ceramic, metal, fiber. Some examples of preferred materials include polysulfone, polypropylene, polyethylene, polyethylene terephthalate, polyethersulfone, polytetrafluoroethylene, cellulose, cellulose acetate, cellulose acetate butyrate, acrylonitrile PVC copolymer, polystyrene, polystyrene/acrylonitrile copolymer, polyvinylidene fluoride, TEFLON and similar materials, ceramic, glass, PEEK, metal, silica, and combinations of the above listed materials.

Some embodiments of the invention employ frits having a low pore volume, which contribute to reducing dead volume. The frits of the invention are porous, since it is necessary for fluid to be able to pass through the frit. The frit should have sufficient structural strength so that frit integrity can contain the chromatography media in the column. It is desirable that the frit have little or no affinity for chemicals with which it will come into contact during the chromatography process, particularly the analyte of interest. In many embodiments of the invention the analyte of interest is a biomolecule, particularly a biological macromolecule. Thus in many embodiments of the invention it desirable to use a frit that has a minimal tendency to bind or otherwise interact with biological macromolecules, particularly proteins, peptides and nucleic acids.

Frits of various pores sizes and pore densities may be used provided the free flow of liquid is possible and the beads are held in place within the chromatography media bed.

In one embodiment, one frit (e.g., a lower frit) is bonded to and extends across the open channel of the column body. In certain embodiments, a second frit is bonded to and extends across the open channel between the bottom frit and the open upper end of the column body (the upper frit). In other embodiments, the upper frit is absent.

In this embodiment, the top frit, bottom frit and column body (i.e., the inner surface of the channel) define a chromatography media chamber wherein a bed of chromatography media is positioned. The frits should be securely attached to the column body and extend across the opening and/or open end so as to completely occlude the channel, thereby substantially confining the bed of chromatography media inside the chromatography media chamber.

In some embodiments of the invention, the bottom frit is located at the open lower end of the column body. This configuration is shown in the figures and exemplified in the Examples, but is not required, i.e., in some embodiments the bottom frit is located at some distance up the column body from the open lower end. However, in view of the advantage that comes with minimizing dead volume or facilitating collection of materials from the column, it is desirable that the lower frit and chromatography media chamber be located at or near the lower end. In some cases this can mean that the bottom frit is attached to the face of the open lower end. However, in some cases there can be some portion of the lower end extending beyond the bottom frit. For the purposes of this invention, so long as the length of this extension is such that it does not substantially introduce dead volume into the chromatography column or otherwise adversely impact the function of the column, the bottom frit is considered to be located at the lower end of the column body.

Frits of the invention can have pore openings or mesh openings of a size in the range of about 5-100 μm, 10-200 μm, or 15-50 μm. In certain embodiments the pore or mesh openings are about 43 μm. The performance of the column is typically enhanced by the use of frits having pore or mesh openings sufficiently large so as to minimize the resistance to flow. The use of membrane screens as described herein typically provide this low resistance to flow and hence better flow rates, reduced back pressure and minimal distortion of the bed of chromatography media. The pore or mesh openings of course should not be so large that they are unable to adequately contain the chromatography media in the chamber.

Some embodiments of the invention employ a thin frit, less than 3.2 mm in thickness, less than 2 mm in thickness, less than 1 mm in thickness (e.g., in the range of 20-350 μm, 40-350 μm, or 50-350 μm), more preferably less than 200 μm in thickness (e.g., in the range of 20-200 μm, 40-200 μm, or 50-200 μm), more preferably less than 100 μm in thickness (e.g., in the range of 20-100 μm, 40-100 μm, or 50-100 μm), and most preferably less than 75 μm in thickness (e.g., in the range of 20-75 μm, 40-75 μm, or 50-75 μm).

Some embodiments of the invention employ a membrane screen as the frit. The membrane screen should be strong enough to not only contain the chromatography media in the column bed, but also to avoid becoming detached or punctured during the actual packing of the media into the column bed. Membranes can be fragile, and in some embodiments must be contained in a framework to maintain their integrity during use. However, it is desirable to use a membrane of sufficient strength such that it can be used without reliance on such a framework. The membrane screen should also be flexible so that it can conform to the column bed. This flexibility is advantageous in the packing process as it allows the membrane screen to conform to the bed of chromatography media, resulting in a reduction in dead volume.

The membrane can be a woven or non-woven mesh of fibers that may be a mesh weave, a random orientated mat of fibers i.e. a “polymer paper,” a spun bonded mesh, an etched or “pore drilled” paper or membrane such as nuclear track etched membrane or an electrolytic mesh (see, e.g., U.S. Pat. No. 5,556,598). The membrane may be e.g., polymer, glass, or metal provided the membrane is low dead volume, allows movement of the various sample and processing liquids through the column bed, may be attached to the column body, is strong enough to withstand the bed packing process, is strong enough to hold the column bed of beads, and does not interfere with the chromatography process i.e. does not adsorb or denature the sample molecules.

The frit may be a fabric, cloth, or sintered material such as polymer, ceramic or metal sintered material or any porous material that can provide the support for the hydrogen bonding of the liquid. This hydrogen bonding of the liquid allows liquid to enter and pass through the column under gravity conditions of the liquid above the low cross sectional area of the bed but does not allow air to enter the bed of the column.

The frit can be attached to the column body by any means which results in a stable attachment such as friction, welding, gluing, or fasteners. For example, the screen can be bonded to the column body through welding or gluing. Gluing can be done with any suitable glue, e.g., silicone, cyanoacrylate glue, epoxy glue, and the like. The glue or weld joint must have the strength required to withstand the process of packing the bed of chromatography media and to contain the chromatography media with the chamber. For glue joints, glue should be employed that does not adsorb or denature the sample molecules.

For example, glue can be used to attach a membrane to the tip of a pipette tip-based chromatography column, i.e., a column wherein the column body is a pipette tip. A suitable glue is applied to the end of the tip. In some cases, a rod may be inserted into the tip to prevent the glue from spreading beyond the face of the body. After the glue is applied, the tip is brought into contact with the membrane frit, thereby attaching the membrane to the tip. After attachment, the tip and membrane may be brought down against a hard flat surface and rubbed in a circular motion to ensure complete attachment of the membrane to the column body. After drying, the excess membrane may be trimmed from the column with a razor blade.

Alternatively, the column body can be welded to the membrane by melting the body into the membrane, or melting the membrane into the body, or both. In one method, a membrane is chosen such that its melting temperature is higher than the melting temperature of the body. The membrane is placed on a surface, and the body is brought down to the membrane and heated, whereby the face of the body will melt and weld the membrane to the body. The body may be heated by any of a variety of means, e.g., with a hot flat surface, hot air or ultrasonically. Immediately after welding, the weld may be cooled with air or other gas to improve the likelihood that the weld does not break apart.

Alternatively, a frit can be attached by means of an annular pip, as described in U.S. Pat. No. 5,833,927. This mode of attachment is particularly suited to embodiment where the frit is a membrane screen.

The frits of the invention, e.g., a membrane screen, can be made from any material that has the required physical properties as described herein. Examples of suitable materials include nylon, polyester, polyamide, polycarbonate, cellulose, polyethylene, nitrocellulose, cellulose acetate, polyvinylidine difluoride, polytetrafluoroethylene (PTFE), polypropylene, polysulfone, metal and glass. A specific example of a membrane screen is the 43 μm pore size Spectra/Mesh® polyester mesh material which is available from Spectrum Labs (Ranch Dominguez, Calif., Part Number 145837).

Pore size characteristics of membrane filters can be determined, for example, by use of method #F316-30, published by ASTM International, entitled “Standard Test Methods for Pore Size Characteristics of Membrane Filters by Bubble Point and Mean Flow Pore Test.”

The polarity of the membrane screen can be important. A hydrophilic screen will promote contact with the bed and promote the air—liquid interface setting up a surface tension. A hydrophobic screen would not promote this surface tension and therefore the threshold pressures to flow would be different. A hydrophilic screen is preferred in certain embodiments of the invention.

### Column Assembly

The columns of the invention can be constructed by a variety of methods using the teaching supplied herein. In some preferred embodiments the column can be constructed by the engagement (i.e., attachment) of upper and lower tubular members (i.e., column bodies) that combine to form the column. Examples of this mode of column construction are described in the Examples and depicted in the figures.

The columns may be assembled or packed into plates or assembled into racks for automated or semiautomated use or they may be individual columns for manual use. In some embodiments of the invention, a column is constructed by the engaging outer and inner column bodies, where each column body has two open ends (e.g., an open upper end and an open lower end) and an open channel connecting the two open ends (e.g., a tubular body, such as a pipette tip). The outer column body has a first frit bonded to and extending across the open lower end, either at the very tip of the open end or near the open end. The section of the open channel between the open upper end and the first frit defines an outer column body. The inner column body likewise has a frit bonded to and extending across its open lower end.

To construct a column according to this embodiment, a chromatography media of interest is disposed within the lower column body, e.g., by orienting the lower column body such that the open lower end is down and filling or partially filling the open channel with the resin, e.g., in the form of a slurry. The inner column body, or at least some portion of the inner column body, is then inserted into the outer column body such that the open lower end of the inner body (where the second frit is attached) enters the outer column body first. The inner column body is sealingly positioned within the open channel of the outer column body, i.e., the outer surface of the inner column body forms a seal with the surface of the open. The section of the open channel between the first and second frits contains the chromatography media, and this space defines a media chamber. In general, it is advantageous that the volume of the media chamber (and the volume of the bed of chromatography media positioned within said media chamber) is less than the outer column body, since this difference in volume facilitates the introduction of chromatography media into the outer column body and hence simplifies the production process. This is particularly advantageous in embodiments of the invention wherein the chromatography columns are mass produced.

In certain embodiments of the above manufacturing process, the inner column body is stably affixed to the outer column body by frictional engagement with the surface of the open channel.

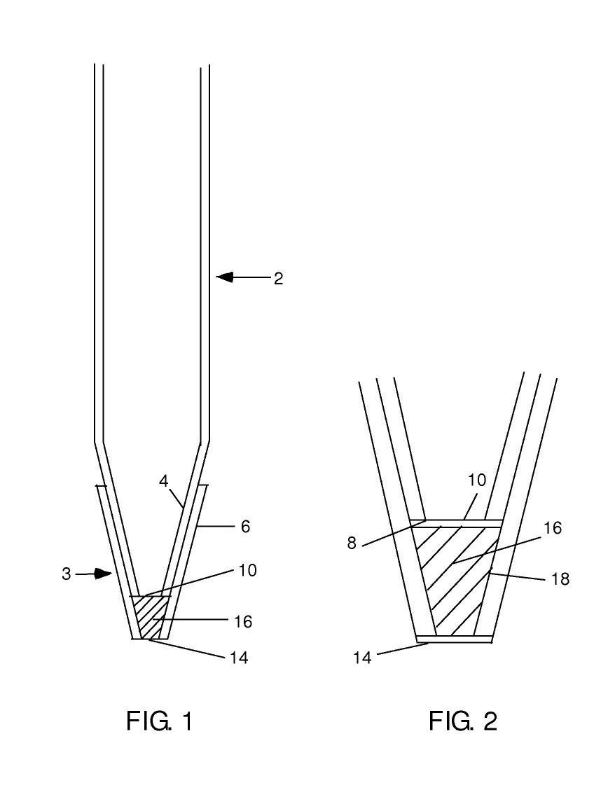

In some embodiments, one or both of the column bodies are tubular members, particularly pipette tips, sections of pipette tips or modified forms of pipette tips. For example, an embodiment of the invention wherein in the two tubular members are sections of pipette tips is depicted in FIG. 1 (FIG. 2 is an enlarged view of the open lower end and chromatography media chamber of the column). This embodiment is constructed from a frustoconical upper tubular member 2 and a frustoconical lower tubular member 3 engaged therewith. The engaging end 6 of the lower tubular member has a tapered bore that matches the tapered external surface of the engaging end 4 of the upper tubular member, the engaging end of the lower tubular member receiving the engaging end of the upper tubular member in a telescoping relation. The tapered bore engages the tapered external surface snugly so as to form a good seal in the assembled column.

A frit 10 is bonded to and extends across the tip of the engaging end of the upper tubular member and constitutes the upper frit of the chromatography column. Another frit 14 is bonded to and extends across the tip of the lower tubular member and constitutes the lower frit of the chromatography column. The chromatography media chamber 16 is defined by the frits 10 and 14 and the channel surface 18, and is packed with chromatography media.

The pore volume of frits 10 and 14 is low to minimize the dead volume of the column. The sample and elution solution can pass directly from the vial or reservoir into the bed of chromatography media. The low dead volume permits elution of the analyte into the smallest possible elution volume, thereby maximizing analyte concentration.

The volume of the chromatography media chamber 16 is variable and can be adjusted by changing the depth to which the upper tubular member engaging end extends into the lower tubular member, as determined by the relative dimensions of the tapered bore and tapered external surface.

The sealing of the upper tubular member to the lower tubular in this embodiment is achieved by the friction of a press fit, but could alternatively be achieved by welding, gluing or similar sealing methods.

Note that in this and similar embodiments, a portion of the inner column body is not disposed within the first channel, but instead extends out of the outer column body. In this case, the open upper end of the inner column body is adapted for operable attachment to a pump, e.g., a pipettor.

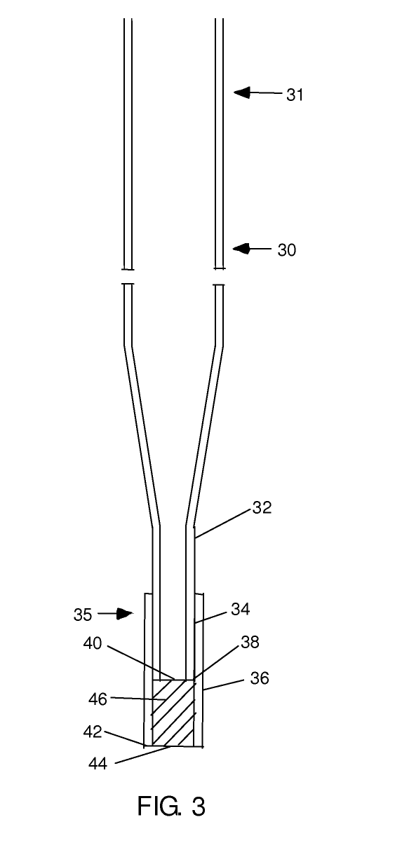

FIG. 3 depicts an embodiment of the invention comprising an upper and lower tubular member engaged in a telescoping relation that does not rely on a tapered fit. Instead, in this embodiment the engaging ends 34 and 35 are cylindrical, with the outside diameter of 34 matching the inside diameter of 35, so that the concentric engaging ends form a snug fit. The engaging ends are sealed through a press fit, welding, gluing or similar sealing methods. The volume of the chromatography bed can be varied by changing how far the length of the engaging end 34 extends into engaging end 35. Note that the diameter of the upper tubular member 30 is variable; in this case it is wider at the upper open end 31 and tapers down to the narrower engaging end 34. This design allows for a larger volume in the column channel above the chromatography media, thereby facilitating the processing of larger sample volumes and wash volumes. The size and shape of the upper open end can be adapted to conform to a pump used in connection with the column. For example, upper open end 31 can be tapered outward to form a better friction fit with a pump such as a pipettor or syringe.

A membrane screen frit 40 is bonded to and extends across the tip 38 of engaging end 34 and constitutes the upper frit of the chromatography column. Another membrane screen frit 44 is bonded to and extends across the tip 42 of the lower tubular member 36 and constitutes the lower frit of the chromatography column. The chromatography media chamber 46 is defined by the membrane screens 40 and 44 and the open interior channel of lower tubular member 36, and is packed with chromatography media.

In other embodiments of this general method of column manufacture, the entire inner column body is disposed within the first open channel. In these embodiments, the first open upper end is normally adapted for operable attachment to a pump, e.g., the outer column body is a pipette tip and the pump is a pipettor. In some preferred embodiments, the outer diameter of the inner column body tapers towards its open lower end, and the open channel of the outer column body is tapered in the region where the inner column body frictionally engages the open channel, the tapers of the inner column body and open channel being complementary to one another. This complementarity of taper permits the two bodies to fit snuggly together and form a sealing attachment, such that the resulting column comprises a single open channel containing the bed of media bounded by the two frits.

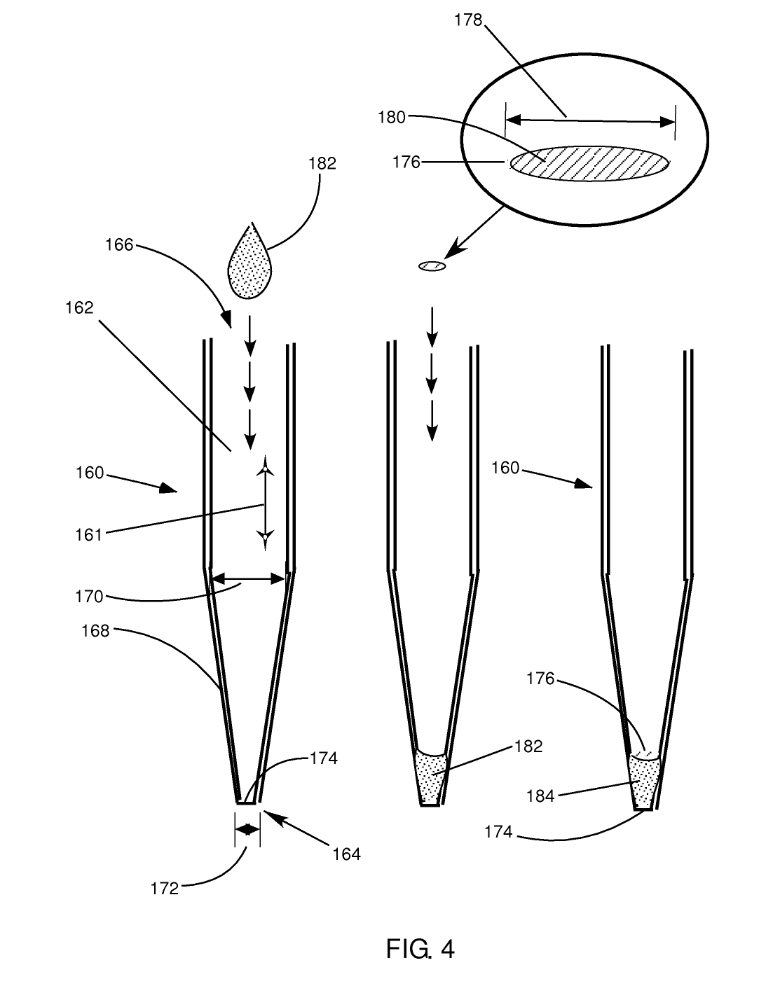

FIG. 4 illustrates the construction of an example of this embodiment of the chromatography columns of the invention. This example includes an outer column body 160 having a longitudinal axis 161, a central through passageway 162 (i.e., an open channel), an open lower end 164 for the expulsion of fluid, and an open upper end 166. The outer column body includes a frustoconical section 168 of the through passageway 162, which is adjacent to the open lower end 164. The inner diameter of the frustoconical section decreases from a first inner diameter 170, at a position in the frustoconical section distal to the open lower end, to a second inner diameter 172 at the open lower end. A lower frit 174, extends across the open lower end 164. In some embodiments, a membrane screen frit can be bound to the outer column body by methods described herein, such as by gluing or welding. This embodiment further includes a ring 176 having an outer diameter 178 that is less than the first inner diameter 170 and greater than the second inner diameter 174. An upper frit 180, extends across the ring.

To construct the column, a desired quantity of chromatography media 182, preferably in the form of a slurry, is introduced into the through passageway through the open upper end and positioned in the frustoconical section adjacent to the open lower end. The chromatography media preferably forms a packed bed in contact with the lower frit 174. The ring 176 is then introduced into the through passageway through the open upper end and positioned at a point in the frustoconical section where the inner diameter of the frustoconical section matches the outer diameter 178 of the ring, such that the ring makes contact with and forms a seal with the surface of the through passageway. The upper frit, lower frit, and the surface of the through passageway bounded by the upper and lower frits define a chromatography media chamber 184. in certain embodiments, the amount of media introduced into the column is selected such that the resulting packed bed substantially fills the chromatography media chamber, preferably making contact with the upper and lower frits. That is, the bed is not tightly packed.

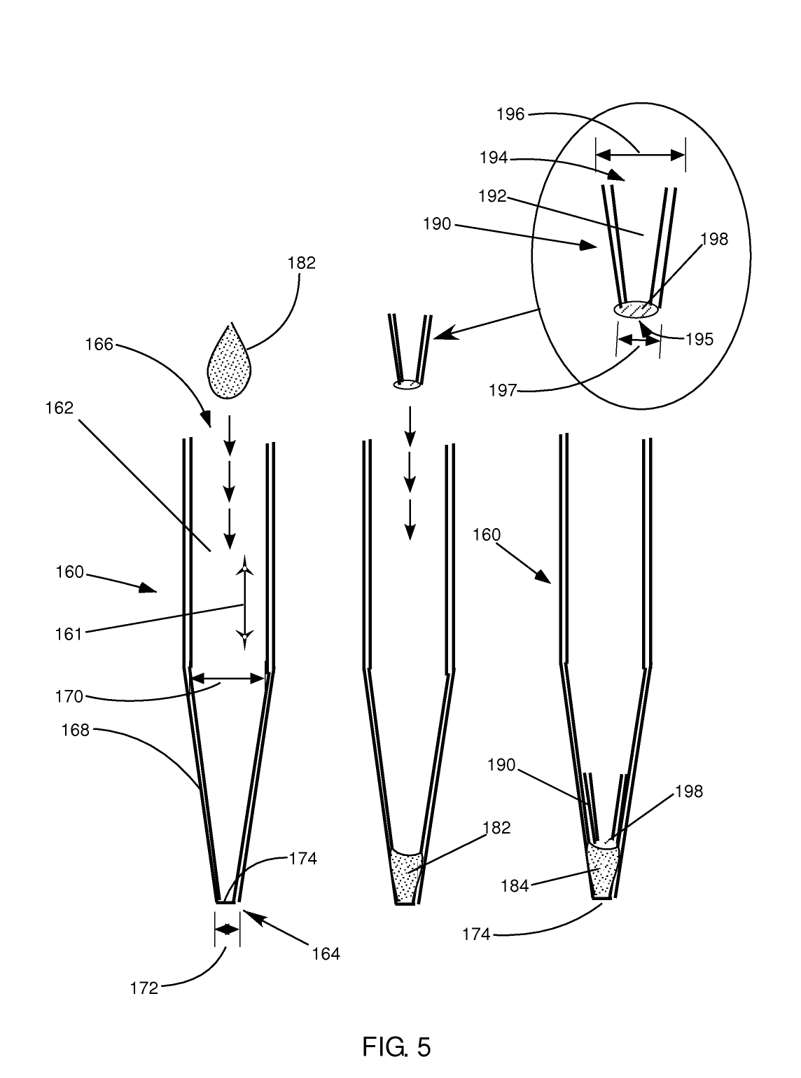

Note that the ring can take any of a number of geometries other than the simple ring depicted in FIG. 4, so long as the ring is shaped to conform to the internal geometry of the frustoconical section and includes a through passageway through which solution can pass. For example, FIG. 5 depicts an embodiment wherein the ring takes the form of a frustoconical member 190 having a central through passageway 192 connecting an open upper end 194 and open lower end 195. The outer diameter of the frustoconical member decreases from a first outer diameter 196 at the open upper end to a second outer diameter 197 at the open lower end. The second outer diameter 197 is greater than the second inner diameter 172 and less than the first inner diameter 170. The first outer diameter 196 is less than or substantially equal to the first inner diameter 170. An upper frit 198 extends across the open lower end 195. Upper frit 198 can be bonded to open lower end 195. The frustoconical member 190 is introduced into the through passageway of an outer column body containing a bed of media positioned at the lower frit 174. The tapered outer surface of the frustoconical member matches and the taper of the frustoconical section of the open passageway, and the two surfaces make a sealing contact. The extended frustoconical configuration of this embodiment of the ring facilitates the proper alignment and seating of the ring in the outer passageway.

Because of the friction fitting of the ring to the surface of the central through passageway, it is normally not necessary to use additional means to bond the upper frit to the column. If desired, one could use additional means of attachment, e.g., by bonding, gluing, welding, etc. In some embodiments, the inner surface of the frustoconical section and/or the ring is modified to improve the connection between the two elements, e.g., by including grooves, locking mechanisms, etc.

In the foregoing embodiments, the ring and latitudinal cross sections of the frustoconical section are illustrated as circular in geometry. Alternatively, other geometries could be employed, e.g., oval, polygonal or otherwise. Whatever the geometries, the ring and frustoconical shapes should match to the extent required to achieve an adequately sealing engagement. The frits are preferably, but not necessarily, positioned in a parallel orientation with respect to one another and perpendicular to the longitudinal axis.

The spacing and arrangement of the multi-channel pipette apparatus or robotic liquid handler of the present invention preferably is complementary to spacing found in existing fluid handling systems, e.g., compatible with multi-well plate dimension. For example, in preferred aspect, the pipettes (or syringes) are positioned or arranged in a linear format (e.g., along a line) or gridded fashion at regularly spaced intervals. For example, in preferred embodiments, the pipettes of the apparatus are arranged on approximately 9 mm centers (96-well plate compatible) in a linear or gridded arrangement, or 4.5 mm centers (384 well plate compatible).

Typically the analyte is a biomolecule and the sample solution containing the analyte is an aqueous solution, typically containing a buffer, salt, and/or surfactants to solubilize and stabilize the biomolecule. In some embodiments, the sample is a biological fluid such as blood, urine, saliva, etc.

The back pressure of a column will depend on the average bead size, bead size distribution, average bed length, average cross sectional area of the bed, back pressure due to the frit and viscosity of flow rate of the liquid passing through the bed. For a 200 uL bed described in this application, the backpressure of columns at 2 mL/min flow rate ranged from 0.5 to 5 psi. For a GE G-25 Sephadex column having bed size of 200 uL, the range was 0.7 psi at a flow rate of 1 ml/min. Other column dimensions will result in backpressures ranging from, e.g., 0.1 psi to 30 psi depending on the parameters described above. Columns with higher backpressures may still be used in this invention although flow purification and processing times may be longer.

In some embodiments, the invention provides columns characterized by small bed volumes, small average cross-sectional areas, and/or low backpressures. This is in contrast to previously reported columns having small bed volumes but having higher backpressures, e.g., for use in HPLC. Examples include backpressures under normal operating conditions (e.g., 2 mL/min in a column with 200 μL bed) less than 30 psi, less than 10 psi, less than 5 psi, less than 2 psi, less than 1 psi, less than 0.5 psi, less than 0.1 psi, less than 0.05 psi, less than 0.01 psi. An advantage of low back pressures is that it allows gravity flow.

Using the force of gravity to drive the solutions through the column. Other technologies having higher backpressures need a higher pressure to drive solution through, e.g., centrifugation at relatively high speed. The gravity force of the liquid above the column is very low because of the low cross-sectional area presented by the column top frit. The cross-sectional area of the top frit is limited because of the 9.0 mm or 4.5 mm center-to-center spacing needed for the columns to be operated on robotic liquid handlers.

Often it is desirable to automate the method of the invention. For that purpose, the subject invention provides a device for performing the method comprising columns containing a packed bed of gel filtration desalting media, placed in a rack in a liquid handler.

The automated means for operating the liquid handler is controlled by software. This software controls the pipettes, and can be programmed to introduce desired liquids into the tops of the gel filtration column using pipette tips as well as to move the rack of columns from position to position to collect aliquots fractions of liquid.

For example, in certain embodiments the invention provides a general method for passing liquid through a rack of packed-bed pipette tip columns comprising the steps of:
a) providing a rack of columns comprising:
   i. a column body having an open upper end for communication with a pump, an open lower end for the uptake and dispensing of fluid, and an open passageway between the upper and lower ends of the column body;
   ii. a bottom frit attached to and extending across the open passageway;
   iii. a top frit attached to and extending across the open passageway between the bottom frit and the open upper end of the column body, wherein the top frit, bottom frit, and surface of the passageway define a media chamber;
   iv. a packed bed of media positioned inside the media chamber;
b) applying liquid aliquots to the top of the rack of columns using robotic liquid handlers and pipettes and liquid passing through the rack of columns by gravity flow
c) collecting liquid aliquots of liquid from the bottom of rack of columns in individual wells or vials.

In certain embodiments, the storage liquid is a water miscible solvent having a viscosity greater than that of water. In certain embodiments the water miscible solvent has a boiling point greater than 250° C. The water miscible solvent can comprise 50%-100% of the storage liquid. In some preferred embodiments the water miscible solvent comprises a diol, triol, or polyethylene glycol of n=2 to n=150, e.g., glycerol.

Packing the chromatography columns is performed in a manner that results in uniform flow. The bed is packed uniformly but not compressed or overly compressed. Every column is different and one column cannot flow exactly same as the other column(s). A slurry of resin is introduced into the column and the resin is settled by pressure, vacuum or gravity. The slurry is made up of gel filtration desalting media that has been swollen overnight or in some cases few days in water or buffer. In some embodiments the slurry is made with water. In other embodiments the slurry is made with a high viscosity solvent to slow the settling of material to facilitate easier packing and more uniform bed volume of the slurry into the column. In other embodiments, the slurry is balanced with a salt or molecular species that makes a high density solvent. Non limiting examples of high density additives include cesium chloride, potassium carbonate, sucrose, glucose, glycerol and propylene glycol.

After the slurry is packed into the column, the frit is placed on top of the bed. Compression of the bed is limited and at least uniform so that the liquid flow through column is uniform. The frit is placed in the column so that there is no air gap between the column bed and frit. In some embodiments, a floating frit is used and then in some cases set into place with wall compression or welding. In other embodiments, the frit at the bottom of the insert is flexible so that when the top frit is positioned into place (see FIG. 5, reference number 190). Low pressure is exerted to the bed of the column and bed compression is limited. In some embodiments, the top frit is spongy and flexible so that when the frit is placed at top of the column the frit is compressed rather than the bed. In some embodiments, there is no top frit. In this case, care must be taken not to disturb the resin bed when sample and chaser aliquots are added.

### Multiplexing

In some embodiments of the invention a plurality of columns is run in a parallel fashion, e.g., multiplexed. This allows for the simultaneous, parallel processing of multiple samples. Multiplexing can be accomplished, for example, by arranging the columns in parallel so that fluid can be passed through them concurrently. Multiplexing is the heart of this invention. Due to the small size of the column, especially the cross sectional area, and the small liquid aliquots applied to the column at the various processing steps, it is difficult to achieve uniform flow through the columns. Uniform flow is achieved by using columns that are uniformly packed and have similar column backpressures, adding liquid uniformly to the top of each column just above the frit so that no air enters the column, using a top frit that stops the flow of liquid when the meniscus of liquid reaches the top of the column, and collecting drop of liquid flow evenly across the columns.

Even with these precautions the method usually has a pause built into the step so that the flow can catch up to the slowest column in the rack or plate. Examples of pause times include 0.5, 1, 2, 5, 10, 15, 17, 20, 25 and 30 minutes. After the pause time has elapsed, all the menisci have reached the top frit. If the top frit is absent, all the menisci have reached the top of the bed of media.

Generally, a certain volume is processed or flowed through a column within a range of time even with some variations of the columns. These parameters include the frit backpressure, cross section area of the column, resin type and compressibility, resin average size, size distribution of the resin, compression of the resin within the column and finally the buffer or liquid that is flowing through the column. For example, 200 mL resin bed gel filtration columns of the invention packed with Sephadex G-25 fine resin can process 600 mL aliquot of water in 8-9 minutes and a 70 mL of water in 1.5-2.5 minutes. However, in another example with the same gel filtration column, using 6M guanidine (a dense buffer) slowed the flow rate or increased the processing time. In this example, to process 70 mL of the 6M guanidine buffer took between 3-5 minutes. A 20 mL aliquot can be processed as quickly as 1 minute and as slow as 5 minutes due to parameters listed above. For a 50 mL aliquot, the aliquot can be processed as quickly as 3 minutes and as slow as 15 minutes again due to the parameters listed above. For a given set of columns and conditions, the flow rates do not vary more than +/−20%, +/−10%, +/−5%, +/−2.5% of the average flow time within the set of columns.

In one embodiment, sample can be arrayed from a chromatography column to a plurality of predetermined locations, for example locations on a chip or micro-wells in a multi-well plate. A precise liquid processing system can be used to dispense the desired volume of eluent at each location. For example, a transfer pipette containing 50 μL of sample or chaser buffer are dispensed into the rack or plate of gel filtration columns using a robotic system such as those commercially available from Zymark (e.g., the SciClone sample handler), Tecan (e.g., the Genesis NPS, Aquarius or TeMo) or Cartesian Dispensing (e.g., the Honeybee bench-top system), Packard (e.g., the MiniTrak5, Evolution, Platetrack. or Apricot), Beckman (e.g., the FX-96) and Matrix (e.g., the Plate Mate 2 or SerialMate). This can be used for high-throughput assays, crystallizations, etc. The term, “liquid handler” is defined herein as any robotic workstation, such as those described above.

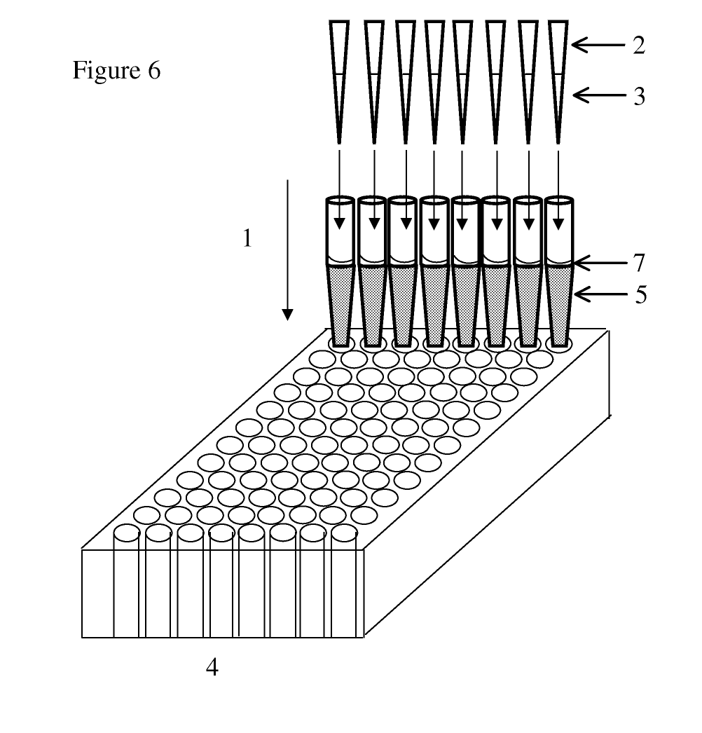

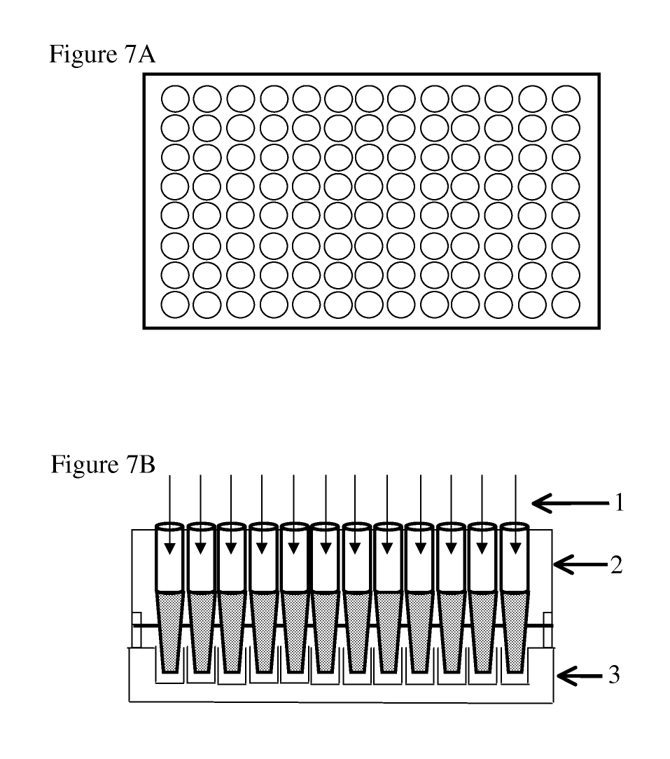

FIGS. 6 and 7 depict examples of a rack of columns used in a multiplexed chromatography system. FIG. 6 shows eight gel filtration desalting columns with collection plate 4. Although the figure text describes gel filtration columns and method, these formats are also used with any chromatography medium. The gel filtration columns can be packed with different types of gel filtration resins into resin bed 5. The liquid/fluid chaser aliquots are added to upper end 1 of the columns by transfer tips 2 with liquid/fluid chaser aliquots 3 and the aliquots are processed in direction 1 by gravity flow. The flow of the liquid stops when liquid meniscus 7 reaches the top frit. The top frit prevents air from entering the resin bed so the column does not dry, crack or channel, which would result in poor performance. The method is paused long enough for the meniscus in each of the columns to reach the top frit. In some embodiments, the top frit is absent, in which case the method is paused long enough for the meniscus in each of the columns to reach the top of the bed. At this point, when liquid flow is stopped for all columns, the next aliquot of liquid is added.

FIG. 7A shows the top view of the 96 gel filtration columns in a rack or plate sitting on top of a collection plate. When columns arranged in the 96-well format are viewed from above, the distance between the centers of two adjacent columns will be 9.0 mm. FIG. 7B shows the side-view of 96 gel filtration columns in rack or plate 2 sitting on top of collection plate 3. 96 gel filtration columns are held in rack or plate 2. The rack/plate serves three purposes. First, it holds 96 gel filtration columns in standard 96-well format. Second, the rack or plate allows the robotic instrument to move 96 columns simultaneously from one position to another. Third, the rack or plate positions the end of the gel filtration columns close to the bottom of the collection plate. The plate is designed to collect all of the eluent that has passed through the column as the liquid/fluid chaser aliquots are added to the open upper end of the columns and processed by gravity flow in the direction indicated by arrows 1.

The robotic liquid handler systems include a controller for pipetting and positioning, columns, plates and racks. The controller is attached to a computer which can be programmed for pipetting control. The controller controls the timing and rate the plunger rack is moved, which in turn is used to control the flow of solution through the columns. The software allows control of the dispensing of aliquots to along with delays between operations.

In some embodiments, the invention provides a multiplexed chromatography system comprising a plurality of chromatography columns of the invention, e.g., gel filtration desalting columns having small beds of packed gel resins. The system can include a pipette, racks and columns in operative engagement with the columns, useful for allowing fluid through the columns in a multiplex fashion, i.e., concurrently. In some embodiments, each column is addressable. The term “addressable” refers to the ability to deliver the fluid individually to each column. An addressable column is one in which the flow of fluid through the column can be controlled independently from the flow through any other column which may be operated in parallel. For example, when pipette pumps are used, then separate transfer tips are used at each column. Because the columns are addressable, a controlled amount of liquid can be accurately manipulated in each column. Various embodiments of the invention can also include samples racks, instrumentation for controlling fluid aliquot manipulation, etc. The controller can be manually operated or operated by means of a computer. The computerized control is typically driven by the appropriate software, which can be programmable, e.g., by means of user-defined scripts.

The invention also provides software for implementing the methods of the invention. For example, the software can be programmed to control manipulation of solutions and addressing of columns into sample vials, collection vials, for spotting or introduction into some analytical device for further processing.

The invention also includes kits comprising one or more reagents and/or articles for use in a process relating to gel filtration, e.g., buffers, standards, solutions, columns, sample containers, etc.

### Consistent Flow in a Column and Across Multiplexed Columns

One the greatest difficulties in achieving consistent flow with a column and across multiplexed gravity flow columns is the prevention of a bubble formation at the head of the column. Liquids are added to the head of the gravity columns with pipette tips or syringe. When adding liquid volumes, the drop or drops of the liquid should cover the complete top of the frit. Preferably no occluded air should be in the liquid above the column after the liquid is added. If there is occluded air is added, it is possible the pocket of air is released by the time the meniscus of the liquid reaches the top of the column. Any air pocket that reaches the frit will reduce the cross sectional area available for gravity to force the liquid through the column. In some cases, this air pocket can cover the entire top of the frit causing the liquid flow to completely stop. This potential problem of air pockets or occluded increases as the diameter of the column decreases and therefore is a problem that is especially difficult for columns and method of use of the invention.

With manual addition of the liquid, visual feedback can be provided to ensure that there are no air pockets added to top of the frit or in the liquid volume above the frit. If air is added, the liquid can be removed and added again. However, when using a liquid handler for the addition of the liquids, there is no opportunity for visual feedback. In this case, the bottom of the transfer pipette tip or needle used for addition of liquids is directed to a position above the frit. In some embodiments, the transfer tip or needle touches the frit. In some embodiments, the lower end of the transfer tip or needle is positioned between 0 and 4 mm of the tip of the column bed. In certain embodiments, the tip is within 3 mm, is within 2 mm and is within 1 mm of the top of the column bed. It is surprising that liquids can be added to multiple columns in parallel from these heights above the column bed and that good column performance can be achieved. All of the columns must be manufactured to have similar bed heights so that the tip or needle comes to the same point for liquid dispersion relative to the top frit of all columns. In some embodiments, the tip or needle is raised as the liquid is transferred or dispersed to the top of the column.

During dispensing of liquids, the speed of dispensing is important. When dealing with small volumes, dispensing at a fast speed is more likely to cause a air pocket/air bubble to form on the side of the columns. In some embodiments, the dispensing speed is between 0.05 mL/min and 1 mL/min. In some embodiments, the dispensing speed is 1 mL/min. In some embodiments, the dispensing speed is 0.5 mL/min, is 0.3 mL/min, is 0.2 mL/min and is 0.1 mL/min. Many liquid handler robotic instruments and pipettes incorporate an air blowout at the end of the dispensing or expulsion step. Sometimes, these air blowouts are called trailing gap. In order to eliminate the air bubble formation, air blowout or trailing gap step should be eliminated. Many times, extra air that is blown out can cause an air pocket to form at the top of the column. The liquid handler is programmed to eliminate any pipette error in picking pick up slightly more volume than needed and dispensing the correct volume. For example, for addition of 70 uL sample, pick up 75 uL and dispense 70 uL. This programming goes beyond the normal programming of a pipettes or liquid handler and may have to written with advanced control or special control of the instrumentation.

### Recovery of Native Proteins

In some embodiments, the chromatography devices and methods of the invention are used to purify proteins that are functional, active and/or in their native state, i.e., non-denatured. This is accomplished by performing the gel filtration desalting process under non-denaturing conditions. Non-denaturing conditions encompass the entire protein separation process. General parameters that influence protein stability are well known in the art, and include temperature (usually lower temperatures are preferred), pH, ionic strength, the use of reducing agents, surfactants, elimination of protease activity, protection from physical shearing or disruption, radiation, etc. The particular conditions most suited for a particular protein, class of proteins, or protein-containing composition vary somewhat from protein to protein.

In one embodiment, the gel filtration desalting process is performed under conditions that do not irreversibly denature the protein. Thus, even if the protein is eluted in a denatured state, the protein can be re-natured to recover native and/or functional protein. In this embodiment, the protein is adsorbed to the chromatography surface under conditions that do not irreversibly denature the protein, and eluting the protein under conditions that do not irreversibly denature the protein. The conditions required to prevent irreversible denaturation are similar to those that are non-denaturing, but in some cases the requirements are not as stringent. For example, the presence of a denaturant such as urea, isothiocyanate or guanidinium chloride can cause reversible denaturation. The eluted protein is denatured, but native protein can be recovered using techniques known in the art, such as dialysis to remove denaturant. Likewise, certain pH conditions or ionic conditions can result in reversible denaturation, readily reversed by altering the pH or buffer composition of the eluted protein.

The recovery of non-denatured, native, functional and/or active protein is particularly useful as a preparative step for use in processes that require the protein to be non-denatured in order for the process to be successful. Non-limiting examples of such processes include analytical methods such as binding studies, activity assays, enzyme assays, X-ray crystallography and NMR.

### Method for Desalting a Sample

In some embodiments, the invention is used to change the composition of a solution in which an analyte is present. An example is the desalting of a sample, where some or substantially all of the salt (or other constituent) in a sample is removed or replaced by a different salt (or non-salt constituent). The removal of potentially interfering salt from a sample prior to analysis is important in a number of analytical techniques, e.g., mass spectroscopy. These processes will be generally referred to herein as “desalting,” with the understanding that the term can encompass any of a wide variety of processes involving alteration of the solvent or solution in which an analyte is present, e.g., buffer exchange or ion replacement.

Desalting and buffer exchange can be accomplished by means of a desalting tip column containing a packed bed of size exclusion media, e.g., a Sephadex G-10, G-15, G-25, G-50 or G-75 resin. Methodology for making and using size exclusion desalting tip columns is provided below in Example 3.

In some embodiments of the above-described procedure, the bed of desalting media is a size exclusion resin, such as Sephadex. This size exclusion media is typically hydrated by passing water or some aqueous solution, e.g., a buffer, through it. In some embodiments, the interstitial space of the bed is substantially full of water or aqueous solution, while in other embodiments liquid is blown out of the interstitial space prior to passing an analyte-containing sample through the bed.

The high molecular weight analyte is typically a high molecular weight biomolecule such as a protein. The low mass chemical entity is typically a salt, ion, or a non-charged low molecular weight molecule component of a buffer or other solution. As a result of passage through the desalting bed, the high molecular weight sample is separated from some, most, or substantially all of the low mass chemical entity, i.e., the sample is desalted. That is, prior to desalting, the sample solution contains high molecular weight analyte and low mass chemical entity at an initial concentration ratio (as calculated by dividing the concentration of high molecular weight analyte by the concentration of low mass chemical entity). After desalting, the product of the process contains either high molecular weight analyte, either substantially free of the low mass chemical entity, or, if there is some low mass chemical entity present, the final concentration ratio (as calculated by dividing the concentration of high molecular weight analyte by the concentration of low mass chemical entity in the eluted sample) is greater than the initial concentration ratio.

In some embodiments, the initial sample solution is eluted directly from a pipette tip column and into the gravity column chromatography bed.

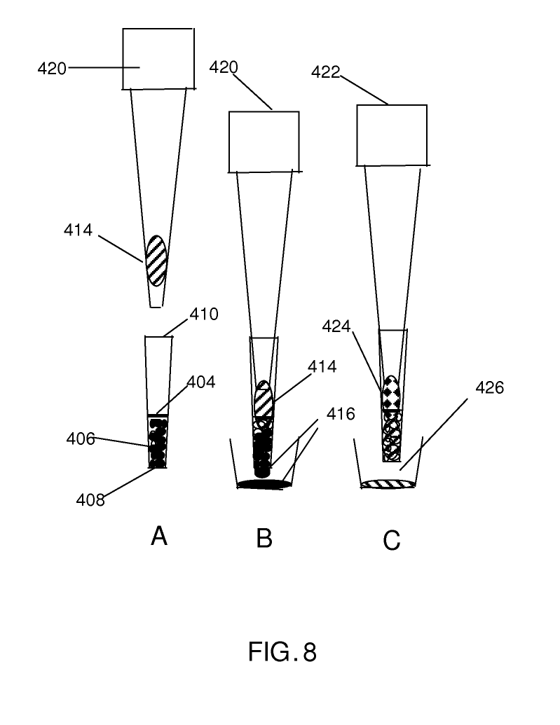

In some embodiments, the analyte is eluted by means of a chaser solution, as described in Example 2 and depicted in FIG. 8.

The uniformity of the gel filtration columns can be measured in terms of Coefficient of Variability (CV). The measurable parameters include volume collected, flow rate, mass of collected molecules, and concentration of molecules in collected volume. After addition of 5 μL to a PhyTip gel filtration column, the collected volume ranges between 4.25-5.7 μL with a CV of 15. Larger volumes will have lower CV values. For collecting volumes of 50 μL the collected volume will range from 46-52 μL with a CV value of 6. In one embodiment, the CV is 10. In another embodiment, the CV is 20. For collecting 10, 20, 50, and 100 μL the CV values range from about 20 to about 5.

The flow rate and collected volume are directly related to the mass and concentration of the target molecule(s) collected provided that the columns are manufactured appropriately. In one embodiment, loading 70 μL of a 2 mg/mL sample of human immunoglobulin G (140 μg total) results in collection of 120-140 μg, with a CV value of 8. In another embodiment, 20 μL of 2 mg/mL samples yields 30-40 μg with a CV value of 14. For dilute or small volume samples containing 5-900 ng, the CV value is 20. For samples containing 1 μg to 500 μg the CV values is 10. For concentrated samples of 600-1000 μg, the CV value is 15. In addition to the column performance, other factors influence the mass recovery. These factors include loss of sample due to too much dilution, or loss of sample due to too much mass, both situations will increase the CV values.

### Analytical Techniques

Chromatography columns and associated methods of the invention find particular utility in preparing samples of analyte for analysis or detection by a variety of analytical techniques. In particular, the methods are useful for purifying an analyte, class of analytes, aggregate of analytes, etc, from a biological sample, e.g., a biomolecule originating in a biological fluid. It is particularly useful for use with techniques that require small volumes of pure, concentrated analyte. In many cases, the results of these forms of analysis are improved by increasing analyte concentration. In some embodiments of the invention the analyte of interest is a protein, and the chromatography serves to purify and concentrate the protein prior to analysis. The methods are particularly suited for use with label-free detection methods or methods that require functional, native (i.e., non-denatured protein), but are generally useful for any protein or nucleic acid of interest.

These methods are particularly suited for application to proteomic studies, the study of protein-protein interactions, and the like. The elucidation of protein-protein interaction networks, preferably in conjunction with other types of data, allows assignment of cellular functions to novel proteins and derivation of new biological pathways. See e.g., Cum Protein Pept. Sci. 2003 4(3):159-81.

Many of the current detection and analytical methodologies can be applied to very small sample volumes, but often require that the analyte be enriched and purified in order to achieve acceptable results. Conventional sample preparation technologies typically operate on a larger scale, resulting in waste because they produce more volume than is required. This is particularly a problem where the amount of starting sample is limited, as is the case with many biomolecules. These conventional methods are generally not suited for working with the small volumes required for these new methodologies. For example, the use of conventional packed bed chromatography techniques tend to require larger solvent volumes, and are not suited to working with such small sample volumes for a number of reasons, e.g., because of loss of sample in dead volumes, on frits, etc. See U.S. patent application Ser. No. 10/434,713 for a more in-depth discussion of problems associated with previous technologies in connection with the enrichment and purification of low abundance biomolecules.

Liquid flow is resisted by the backpressure of the column and by surface tension effects within the column, particularly in the bed and at the interface of the bed and frits. Surface tension or similar force can arise from the interaction of liquid with the packed bed of media and/or with the frit. This force results in an initial resistance to flow of liquid through the bed of chromatography media, described elsewhere herein as a form of “bubble point.” As a result, a certain minimum threshold of head pressure must be generated before liquid will commence flowing through the bed. In addition, there is the backpres sure of the column that must be overcome in order for liquid to flow through the bed. Thus, in operation of the column a sufficiently negative head pressure must be generated to overcome backpressure and other effects prior to flow commencing through the bed. The magnitude of the pressure drop across the column will to some extent depend upon the backpres sure which in turn depends upon the size of the bed, the nature of the media, the nature of the packing, the nature of the frits, and the interaction of the frits with the bed.

During the course of using the columns of the invention, the pressure drop of any given column will vary during the course of the process. As the volume above the head of the column decreases, head pressure for will decrease. For example, let us consider an embodiment where multiple pipette tip columns and a programmable multi-channel pipettor are used.

The pressure drop present at any given step in the separation process will vary from column to column. This variation can be the result of any of a number of factors, including the slight variations from column to column, reflecting subtle difference in the packing of the bed. This can be the case where multiple columns are run sequentially (in series). This can also be the case when multiple columns are run concurrently and/or in parallel. Because of subtle differences from tip to tip, different head pressures can develop from tip to tip. It is surprising that a method can be performed adding the sample, elution solvents at the same time for multiple columns.

In certain embodiments, the invention provides methods of addressing the problems associated with the above-described variations in head pressure.

### Maintaining Pipette Tip Columns and Polymer Beads in a Wet State

In certain embodiments, the invention provides methods of storing pipette tip columns in a wet state, i.e., with a “wet bed” of chromatography media. This is useful in it allows for preparing the columns and then storing for extended periods prior to actual usage without the bed drying out, particularly where the chromatography media is based on a resin, such as a gel resin. For example, it allows for the preparation of wet columns that can be packaged and shipped to the end user, and it allows the end user to store the columns for a period of time before usage. In many cases, if the bed were allowed to dry, out it would adversely affect column function, or would require a time-consuming extra step of re-hydrating the column prior to use.

The maintenance of a wet state can be particularly critical wherein the bed volume of the packed bed is small, e.g., in a range having a lower limit of, 20 μL, or 40 μL, and an upper limit of 50 μL, 100 μL, 200 μL, 300 μL, 500 μL, 1 mL, 2 mL, 5 mL. Typical ranges would include 200 to 2000 μL.

The wet tip results from producing a tip having a packed bed of media wherein a substantial amount of the interstitial space is occupied by a liquid. Substantial wetting would include beds wherein at least 25% of the interstitial space is occupied by liquid, and preferably at least 50%, 70%, 80%, 90%, 95%, 98%, 99%, or substantially the entire interstitial space is occupied by liquid. The liquid can be any liquid that is compatible with the media, i.e., it should not degrade or otherwise harm the media or adversely impact the packing. Preferably, it is compatible with purification and/or chromatography processes intended to be implemented with the column. For example, trace amounts of the liquid or components of the liquid should not interfere with solid phase chromatography chemistry if the column is intended for use in a solid phase chromatography. Examples of suitable liquids include water, various aqueous solutions and buffers, and various polar and non-polar solvents described herein. The liquid might be present at the time the column is packed, e.g., a solvent in which the chromatography media is made into a slurry, or it can be introduced into the bed subsequent to packing of the bed.

In certain embodiments, the liquid is a solvent that is water miscible and that is relatively non-volatile and/or has a relatively high boiling point (and preferably has a relatively high viscosity relative to water). A “relatively high boiling point” is generally a boiling point greater than 100° C., and in some embodiments of the invention is a boiling point at room temperature in range having a lower limit of 100° C., 110° C., 120° C., 130° C., 140° C., 150° C., 160° C., 170° C., 180° C., 190° C., 200° C., or higher, and an upper limit of 150° C., 160° C., 170° C., 180° C., 190° C., 200° C., 220° C., 250° C., 300° C., or even higher. Illustrative examples would include alcohol hydrocarbons with a boiling point greater than 100° C., such as diols, triols, and polyethylene glycols (PEGs) of n=2 to n=150 (PEG-2 to PEG-150), PEG-2 to PEG-20, 1,3-butanediol and other glycols, particularly glycerol and ethylene glycol. The water miscible solvent typically constitutes a substantial component of the total liquid in the column, wherein “a substantial component” refers to at least 5%, and preferably at least 10%, 20%, 30%, 40%, 50%, 60%, 70%, 80%, 90%, 95%, 98%, 99%, or substantially the entire extent of the liquid in the column.

An advantage of these non-volatile solvents is that they are much less prone to evaporate than the typical aqueous solutions and solvents used in chromatography processes. Thus, they will maintain the bed in a wet state for much longer than more volatile solvents. For example, an interstitial space filled with glycerol will in many cases stay wet for days without taking any additional measures to maintain wetness, while the same space filled with water would soon dry out. These solvents are particularly suitable for shipping and storage of gel type resin columns having agarose or sepharose beds. Other advantageous properties of many of these solvents, is that they are viscous so they are not easily displaced from the column during shipping vibrations and movement. In addition, they are bacterial resistant; they do not appreciably penetrate or solvate agarose, sepharose, and other types of packing materials, and they stabilize proteins. Glycerol in particular is a solvent displaying these characteristics. Note that any of these solvents can be used neat or in combination with water or another solvent, e.g., pure glycerol can be used, or a mixture of glycerol and water or buffer, such as 50% glycerol or 75% glycerol.

One advantage of glycerol is that its presence in small quantities has negligible effects on many solid-phase chromatography processes. A tip column can be stored in glycerol to prevent drying, and then used in a chromatography process without the need for an extra step of expelling the glycerol. Instead, a sample solution (typically a volume much greater than the bed volume, and hence much greater than the volume of glycerol) is loaded directly on the column by drawing it up through the bed and into the head space as described elsewhere herein. The glycerol is diluted by the large excess of sample solution, and then expelled from the column along with other unwanted contaminants during the loading and wash steps.

Note that relatively viscous, non-volatile solvents of the type described above, particularly glycerol and the like, are generally useful for storing polymer beads, especially the resin beads as described herein, e.g., agarose and sepharose beads and the like. Other examples of suitable beads would include xMAP® technology-based microspheres (Luminex, Inc., Austin, Tex.). Storage of polymer beads as a suspension in a solution comprising one or more of these solvents can be advantageous for a number of reasons, such as preventing the beads from drying out, reducing the tendency of the beads to aggregate, and inhibiting microbial growth. The solution can be neat solvent, e.g., 100% glycerol, or a mixture, such as an aqueous solution comprising some percentage of glycerol. The solution can also maintain the functionality of the resin bead by maintaining proper hydration, and protecting any affinity binding groups attached to the bead, particularly relatively fragile functional groups, such as certain biomolecules, e.g., proteins.

Factors that can affect the rate at which a column dries include the ambient temperature, the air pressure, and the humidity. Normally columns are stored and shipped at atmospheric pressure, so this is usually not a factor that can be adjusted. However, it is advisable to store the columns at lower temperatures and higher humidity in order to slow the drying process. Typically the columns are stored under room temperature conditions. Room temperature is normally about 25° C., e.g., between about 20° C. and 30° C. In some cases it is preferable to store the pipette tip columns at a relatively low temperature, e.g., between about 0° C. and 30° C., between 0° C. and 25° C., between 0° C. and 20° C., between 0° C. and 15° C., between 0° C. and 10° C., or between 0° C. and 4° C. In many cases, tips of the invention may be stored at even lower temperatures, particularly if the tip is packed with a liquid having a lower freezing point than water, e.g., glycerol.

In one embodiment, the invention provides a pipette tip column that comprises a bed of media, the interstitial space of which has been substantially full of liquid for at least 24 hours, for at least 48 hours, for at least 5 days, for at least 30 days, for at least 60 days, for at least 90 days, for at least 6 months, or for at least one year. “Substantially full of liquid” refers to at least 25%, 50%, 70%, 80%, 90%, 95%, 98%, 99%, or substantially the entire interstitial space being occupied by liquid, without any additional liquid being added to the column over the entire period of time. For example, this would include a column that has been packaged and shipped and stored for a substantial amount of time after production.

In one embodiment, the invention provides a packaged pipette tip column packaged in a container that is substantially full of liquid, wherein the container maintains the liquid in the pipette tip to the extent that less than of 10% of the liquid is (or will be) lost when the tip is stored under these conditions for at least 24 hours, for at least 48 hours, for at least 5 days, for at least 30 days, for at least 60 days, for at least 90 days, for at least 6 months, or for at least one year.

In another embodiment, the invention provides a pipette tip column that comprises a bed of media, the interstitial space of which is substantially full of liquid, wherein the liquid is escaping (e.g., by evaporation or draining) at a rate such that less than 10% of the liquid will be lost when the column is stored at room temperature for 24 hours, 48 hours, 5 days, 30 days, 60 days, 90 days, six months or even one year.

In many cases, the wet pipette tip columns described above (e.g., the column that has been wet for an extended period of time and/or the column that is losing liquid only at a very slow rate) is packaged, e.g., in a pipette tip rack. The rack is a convenient means for dispensing the pipette tip columns, and for shipping and storing them as well. Any of a variety of formats can be used; racks holding 96 tips are common and can be used in conjunction with multi-well plates, multi-channel pipettors, and robotic liquid handling systems.

All publications and patent applications mentioned in this specification are herein incorporated by reference to the same extent as if each individual publication or patent application was specifically and individually indicated to be incorporated by reference.

Having now generally described the invention, the same will be more readily understood through reference to the following examples, which are provided by way of illustration, and are not intended to be limiting of the present invention, unless so specified.

### EXAMPLES

The following preparations and examples are given to enable those skilled in the art to more clearly understand and practice the present invention. They should not be construed as limiting the scope of the invention, but merely as being illustrative and representative thereof.

### Example 1

### Preparation of Gravity Chromatography Column Bodies from Pipette Tips

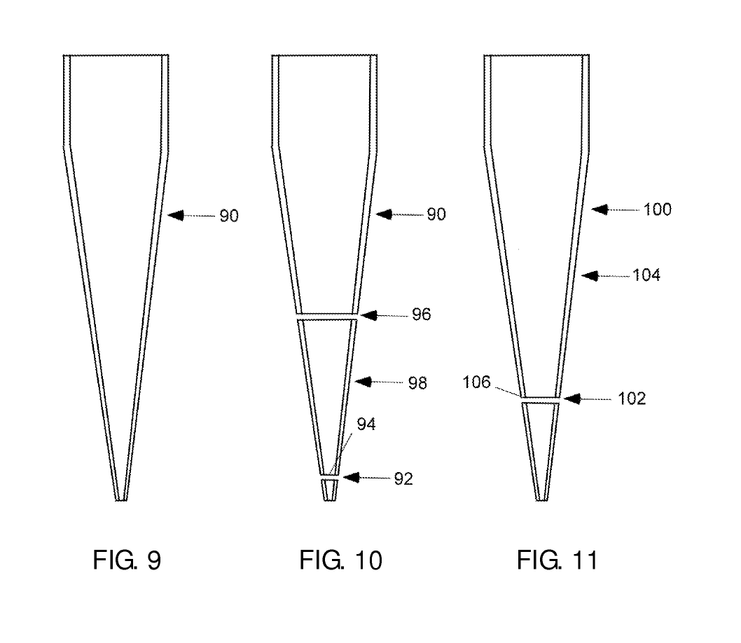

One 200 μL and two 1000 μL polypropylene pipette tips of the design shown in FIG. 9 (Rainin, Alameda, Calif., PN RT-L250W, RT-L1000W and RT-L1200) were used to construct four chromatography columns. In this example, four columns were constructed: an 80 μL bed in a 200 μL column and 200, 600 and 900 μL bed volumes in a 1000 μL column. To construct a column, various components were made by inserting the tips into several custom aluminum cutting tools and cutting the excess material extending out of the tool with a razor blade to give specified column lengths and diameters.

Referring to FIG. 10, the first cut 92 was made to the tip of a pipette tube 90 to form a 3 mm, 4.57 mm, 4.57 mm and 6 mm outside diameter hole 94 on the lower column body, which corresponds to the 80, 200, 600 and 900 μL columns, respectively. A second cut 96 was made to form an inner column body segment 98 having a length of 3.0 mm, 10.0 mm, 7.0 mm, and 9.5 mm, respectively.

Referring to FIG. 11, a cut 102 was made to a pipette tip 100 to form the outer column body 104. To make a 80 μL bed volume column, the cut 102 was made to provide a tip 106 outside diameter of 2.08 mm so that when the inner column body 98 was inserted into the outer body 104, the column height of the solid phase media bed 114 (FIG. 13) was 19 mm. To make a 200 μL bed volume column, the cut 102 was made to provide a tip 106 outside diameter of 5.11 mm so that when the inner column body 98 was inserted into the outer body 104, the column height of the solid phase media bed 114 (FIG. 13) was 13 mm. To make a 600 μL bed volume column, the cut 102 was made to provide a tip 106 outside diameter of 3.86 mm so that when the inner column body 98 was inserted into the outer body 104, the column height of the solid phase media bed 114 (FIG. 13) was 31 mm. To make a 900 μL bed volume column, the cut 102 was made to provide a tip 106 outside diameter of 3.86 mm so that when the inner column body 98 was inserted into the outer body 104, the column height of the solid phase media bed 114 (FIG. 13) was 44.5 mm.

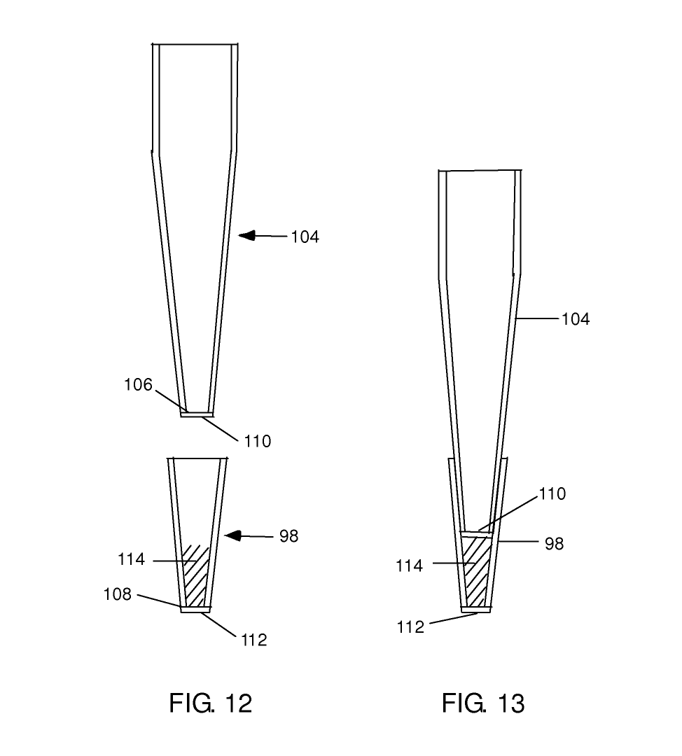

Referring to FIGS. 12 and 13, a 43 μm pore size Spectra/Mesh® polyester mesh material (Spectrum Labs, Ranch Dominguez, Calif., PN 145837) was cut into discs by a circular cutting tool (Pace Punches, Inc., Irvine, Calif.) and attached to the ends 106 and 108 of the inner column and outer column bodies to form the membrane screens 110 and 112. The membrane screens were attached using PLASTIX® cyanoacrylate glue (Loctite, Inc., Avon, OH). The glue was applied to the polypropylene body and then pressed onto the membrane screen material. Using a razor blade, excess mesh material was removed around the outside perimeter of each column body end.

Referring to FIG. 13, the inner column body 104 is inserted into the top of the lower column body segment 98 and pressed downward to compact the solid phase media bed 114 to eliminate excess dead volume above the top of the bed.

### Example 2

### Desalting a Protein Sample of Imidazole by Size Exclusion

A method and apparatus for desalting a protein sample by size exclusion is depicted in FIG. 8. A desalting tip column is prepared using the methodology provided herein in Example 1. The chromatography column 406 is about 100 μL and is packed with a size exclusion media suitable for desalting a protein of interest, e.g., Sephadex G-10, G-15, G-25, G-50 or G-75 (Amersham Biosciences, Piscataway, N.J.). The specific size exclusion media employed will vary depending upon such factors as the size of the protein to be desalted, the nature of constituents of the solution to be desalted, and requirements such as desired speed of the process, yield of product, concentration of product, degree of desalting, etc., as can be determined by one of skill in the art based on the known properties of size exclusion media such as Sephadex.

The size exclusion resin is hydrated with water, or optionally with a buffer such as PBS. Prior to beginning the actual desalting procedure, the bed of size exclusion media may be conditioned again with water or a buffer. The conditioning liquid flows through the column and the flow pauses as the meniscus of the liquid reaches the top of the column.

Referring to FIG. 8A, pipette tip 420 containing a 10 μL drop 414 of purified sample of 100 μg His-tagged protein and 500 mM imidazole is positioned over desalting column 410 comprised of size exclusion medium 406. Also shown are top frit 404 and bottom frit 408.

The upper end of the pipette tip 420 is attached to a pipettor (not shown), and this pipettor is activated to drive the 10 μL of sample 414 out of the tip and onto the top of the bed of size exclusion media (FIG. 8B). Drop 414 is stylized for illustrative purposes and does not show the typical shape of the meniscus. The 10 uL sample flows into the column until the meniscus reaches the top of the bed and then stops. As the 10 uL of sample flows into the column 10 uL of liquid in the column flows out (reference no. 416).

The tip is then removed, and another pipette tip 422 containing 40 μL of chaser elution solution 424 (typically water, or a buffer such as PBS) is inserted into the open upper end of the extraction tip column. The pipette tip 422 is positioned such that the lower end of the pipette tip is close to the top of the bed of size exclusion medium (FIG. 8C). The upper end of the chaser tip is attached to a pipettor (not shown) which is activated to expel the chaser elution solution to the top the bed of size exclusion medium. Desalted His-tagged protein 426 is eluted from the column and collected while the imidazole remains on the column.

In an alternative embodiment, the desalting column can be made according to the design depicted in FIGS. 1 and 2, according to the methodology accompanying those figures. The bed volume is still 100 uL, but the dimensions of the bed are generally wider and shorter.

In another alternative embodiment of the desalting method, 45 μL of elution buffer 424 is used instead of 40 μL to optimize the recovery of the protein.

In other embodiments using different chromatography methods (non-gel filtration) in which the materials of interest are adsorbed or partition, larger elution volumes are generally used to elute the material of interest.

### Example 3

### Automation of the PhyTip Gel Filtration Column

PhyTip gel filtration columns (PhyNexus, Inc., San Jose, Calif.) are compatible with use on the PhyNexus MEA Personal Purification System and the Beckman Biomek FX. With some modification, the columns can be made compatible with most 96-channel liquid handling instruments. Four steps are required for use of the PhyTip gel filtration columns for size-based separations. These steps are column equilibration, column conditioning, sample loading and collection of target molecule(s).

PhyTip column equilibration. The PhyTip columns are shipped with glycerol, which acts as a preservative and prevents the media from dehydrating. The glycerol needs to be removed prior to use of the columns. To remove the glycerol, the end of the PhyTip columns are submerged in buffer such as water supplemented with 0.01% sodium azide to act as a preservative. 1 mL of this buffer is added to the top of the columns and these are allowed to equilibrate for at least eight hours overnight. If the glycerol removal step requires faster processing, then the equilibration step can be performed at 42° C. because the glycerol will be less viscous at higher temperatures. Failure to remove the glycerol will result in glycerol contamination in the final, purified sample fractions, or broadening of the target peaks.

PhyTip column conditioning. Once the glycerol has been removed, the PhyTip gel filtration columns are conditioned and the equilibration buffer in the column is exchanged for the final buffer in which the molecule(s) of interest will be collected. The columns are removed from submersion in the equilibration buffer and suspended over a waste collection reservoir and the residual equilibration buffer is allowed to drain out of the column. As the buffer reaches the top frit screen above the resin bed, the fluid flow will stop. Three column volumes of conditioning buffer is added to the top of the PhyTip gel filtration column and the buffer is allowed to drain until all of the buffer has completely entered the resin bed. The flow is generally even but not perfectly so. The flow of liquid stops when the liquid meniscus reaches the frit, then the flow stops. The top frit screen prevents air from entering the resin bed so that column does not dry, crack or channel, which would result in poor performance. The method is paused long enough for all of the columns to reach this state. At this point liquid flow is stopped for all columns until the next aliquot of liquid is added.

PhyTip column sample loading. The PhyTip columns are ready for injection of the sample. The PhyTip columns are transferred to an apparatus that suspends the ends of the columns inside individual collection wells 4 mm above the bottom of the well. Sample is added to the top of the PhyTip column and allowed to enter the resin bed, completely. Every time sample and buffer enters the resin bed, the meniscus of the fluid will stop when it reaches the top frit. The Resin bed will not go dry and the columns are ready for the next buffer addition. The flow through is collected in the well. Table 1 below describes the injection volume range for different PhyTip columns.

Sample collection. Chaser buffer is added to elute the target molecule(s) from the column. The chaser buffer should be the same composition as the conditioning buffer and will be the final desired buffer. The PhyTip columns are moved to a new collection plate and chaser buffer is added to the top of the PhyTip columns. Multiple volumes of the chaser buffer can be added to the columns in a stepwise fashion and each addition can be collected separately to perform fractionation of the samples. This would require moving the columns to a new collection plate prior to the addition of each new chaser fraction. If buffer exchange is the goal, a larger chaser volume is added to the top of the PhyTip column and the target molecule(s) are collected. Care should be taken that the chaser fraction is not too large so as to release the small molecules that are retained in the gel filtration matrix. To efficiently collect the fractions, the PhyTip columns should be suspended an optimal distance above the bottom of the collection well. As the fluid leaves the PhyTip column, it will form a drop attached at the end of the column. The release of the drop is accomplished by having the drop touch the bottom of the well. Once the column is lifted out of the collection plate, the drop will release. Table 1, below shows the suggested chase volumes to be used with different sample volumes and column sizes for buffer exchange and desalting.

TABLE 1
Suggested sample and chaser volumes

| Column bed volume (μL) | Sample volume (μL) | Chaser volume (μL) |
|---|---|---|
| 200 | 20 | 150 |
| 200 | 30 | 140 |
| 200 | 40 | 130 |
| 200 | 50 | 120 |
| 200 | 60 | 110 |
| 200 | 70 | 100 |
| 200 | 80 | 90 |
| 200 | 90 | 80 |
| 600 | 100 | 400 |
| 600 | 200 | 300 |
| 600 | 300 | 200 |
| 600 | 400 | 100 |

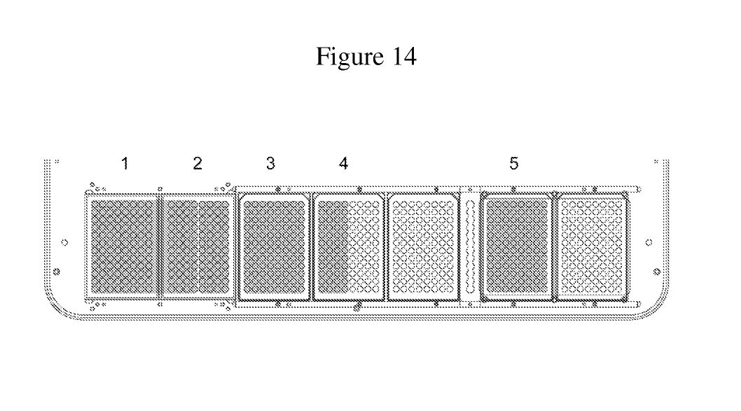

The steps described above can be fully automated. FIG. 14 shows the MEA setup of gel filtration columns for buffer exchange and desalting. The bottom of the figure corresponds to the front of the unit and the top of the figure corresponds to the back of the instrument. 144, 1-mL transfer tips were placed into Position 1 and rows 1-4 of Position 2 (FIG. 14). Forty-eight, 200-μL gel filtration columns were placed into Position 2. A 96-well plate with 0.5-mL capacity in each well was placed in Position 3 and served as a collection plate. Position 4 contained a 2-mL deep-well plate with 1 mL of conditioning buffer in rows 1-4 (FIG. 14, 4). Position 7 was affixed with a rack to maintain the rigidity of a 96-well PCR plate, which was placed on top (FIG. 14, 5). Rows 1-4 contained 20-90 μL of samples 1-48 and rows 5-8 contained 20-90 μL (FIG. 14, 5). The MEA added 600 μL of conditioning buffer to the top of 12 columns and paused 15 minutes for the conditioning buffer to flow through the columns into waste. The MEA then transferred 70-μL samples to the top of the 12 columns and paused 5 minutes for the flow-through to collect into waste. The MEA transferred 120 μL of chaser to the top of the 12 columns. The instrument immediately engaged the columns and moved them to row 1 of the collection plate and held them suspended 4 mm above the bottom of the collection well for 10 minutes. This completed the buffer exchange of samples 1-12 and the MEA repeated the process for the next 12 samples until all 48 samples were processed.

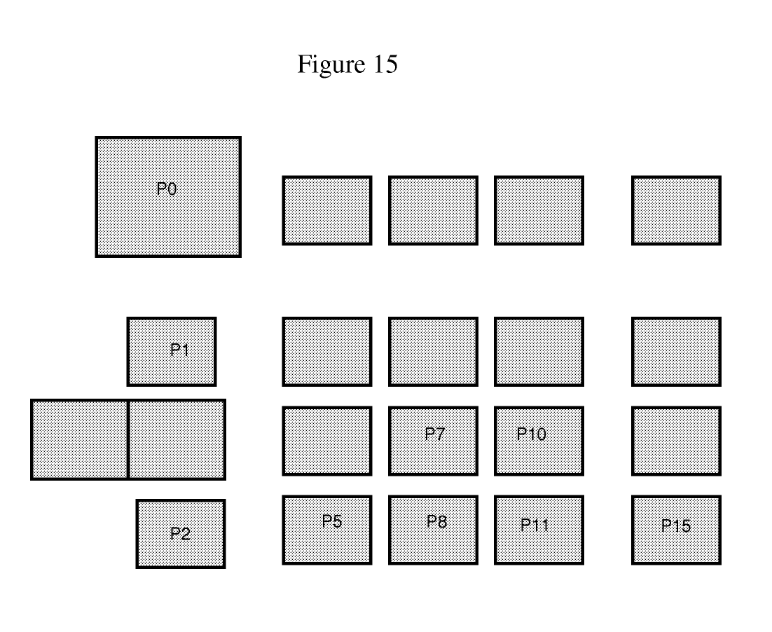

The Beckmam Biomek FX was set up to perform 96 size-based separations using 200 μL gel filtration columns. FIG. 15 shows how to set up a Beckman Biomek FX for use with Gel filtration columns. A box of pipette tips was placed in the tip loader (Position P0) and an additional two boxes was placed at positions (P1) and (P2). The columns were placed into a rack suspended over a waste collection plate in Position (P5). The rack was made specifically for the Biomek FX. It was designed to hold 96 gel filtration columns, serve as a handle for the Biomek FX gripper function to allow all 96 columns to be moved from one deck position to another, and suspends the columns at the proper position above the bottom of the collection well. Position (P11) contained a reservoir plate with 90 mL of conditioning buffer. Position (P7) held a 96-well plate containing 96 70 μL Samples. Position (P10) held a 96-well plate containing 120 μL Chaser Buffer in each well. Position (P5) held a 96-well collection plate. The Biomek FX added 600 μL conditioning buffer to the top of the columns and the instrument paused for 15 minutes while the conditioning buffer flowed through the resin bed and into the waste collection plate. The instrument next added 70 μL sample to each column and the flow through was collected to waste during a 5 minute pause. The instrument moved the columns to the collection plate by employing the gripper function. The instrument added 120 μL chaser to the top of the columns and the flow through was collected.

If fractionation is desired, a stack of collection plates are placed in position (P15). The Biomek FX can take plates from this position and placed them on top of other collection plates at Potion (P5). The rack containing the columns can be stacked on top of these empty plates and serve as collection plates for the desired number of samples.

### Example 4

### Separation of Myoglobin Protein from DNP-Glutamate for Desalting

200-1 μL gel filtration columns were equilibrated overnight and conditioned with 700 μL of PBS buffer (10 mM phosphate, 140 mM NaCl, pH 7.4). 20 μL of sample containing brown 2.4 mM myoglobin protein (16,700 MW) and 3.5 mM DNP-glutamate salt (313 MW) was loaded onto the gel filtration columns. The flow through was collected and the columns were chased with 80 μL PBS buffer. The collected fraction was analyzed using a UV spectrometer to calculate protein recovery and salt removal. Myoglobin protein is brown and has a molar extinction coefficient at 409 nm of 2,700 M⁻¹ cm⁻¹. DNP-glutamate is yellow and has a molar extinction coefficient at 364 nm of 487 M⁻¹ cm⁻¹. The concentration of myoglobin and DNP-glutamate was determined using the equation, c=A/εL, where C is the concentration, A is the absorbance, ε is the molar extinction coefficient, and L is the path length (Table 2).

TABLE 2
Myoglobin recovery and salt removal

| | | | Vol. | pmol | pmol DNP- | % myoglobin | % DNP-glutamate |
|---|---|---|---|---|---|---|---|
| | A₃₆₄ | A₄₀₉ | (μL) | myoglobin | glutamate | recovery | removal |
| Myoglobin input | | 1.165 | 20.0 | 47,843.9 | | | |
| Myoglobin sample 1 | | 0.205 | 90.5 | 38,095.5 | | 79.6 | |
| Myoglobin sample 1 | | 0.200 | 94.8 | 38,932.2 | | 81.4 | |
| DNP-glutamate input | 2.440 | | 20.0 | | 70,469.3 | | |
| DNP-glutamate sample 1 | 0.003 | | 88.7 | | 96.1 | | 99.9 |
| DNP-glutamate sample 1 | 0.006 | | 89.3 | | 193.4 | | 99.7 |

### Example 5

### Recovery of Different Proteins and Optimization of Gel Filtration Columns

Different molecules have properties, namely shape and molecular weight, which differentiates how they interact with the gel filtration column. To determine the appropriate chaser volume to recover a target molecule, it is appropriate to perform a recovery experiment with known standards. 200-μL columns were equilibrated and conditioned as in Example 2. 20 μL samples, 3.1 mg/mL final concentration, of human IgG (human IgG, Sigma-Aldrich) spiked into PBS buffer containing 0.05% Tween, was applied to the top of each column. After the sample entered the resin bed, 120 μL PBS buffer was applied to the column to release the human IgG. The sample flow through and chaser was collected and weighed by an analytical scale and measured by HPLC (Table 3).

TABLE 3
IgG recovery

| | Rec. vol. (μL) | A280 | uM | pmoles | % Recovery |
|---|---|---|---|---|---|
| Input | 20.0 | 0.7 | 3.1 | 62.1 | |
| hIgG sample 1 | 133.4 | 0.1 | 0.3 | 45.7 | 73.7 |
| hIgG sample 3 | 110.0 | 0.1 | 0.4 | 41.9 | 67.5 |

### Example 6

### Sample Collection Reproducibility

The efficient collection of the small drops is very important for the performance of the gel filtration columns. These small volumes are potentially highly concentrated with the molecule(s) of interest. Procedures were developed to ensure reproducibility in volume recovery. Four columns were equilibrated and conditioned as in Example 2. 120 μL PBS was loaded to the top of each column and the flow through was collected. The volume collected was measured by weighing on an analytical scale (Table 4).

TABLE 4
Volume recovery reproducibility

| | Day 1 | Day 2 | Day 3 |
|---|---|---|---|
| Column # | Rec. vol. (μL) | Rec. vol. (μL) | Rec. vol. (μL) |
| 1 | 122.6 | 118.8 | 133.4 |
| 2 | 132.6 | 106.5 | 121 |
| 3 | 112.6 | 119.4 | 110 |
| 4 | 115.0 | 120.6 | |
| Average | 120.7 | 116.3 | 121.5 |
| Standard Deviation | 9.0 | 6.6 | 11.7 |
| CV | 7.5 | 5.7 | 9.6 |

### Example 7

### Column Reproducibility

The columns were tested for reproducibility by measuring the recovery of a standard protein spiked into PBS buffer containing 0.05% Tween 20. Twelve, 200-μL gel filtration columns were equilibrated and conditioned as described in Example 2. 40 μL aliquots of a 2 mg/mL IgG sample were added to the top of the columns and the flow through was discarded. The IgG was released by a chaser buffer of 130 μL PBS. The chaser buffer was collected and analyzed by a UV-spectrometer to quantify the sample recovery (Table 5).

TABLE 5
Gel filtration column performance reproducibility

| | Vol. | [IgG] | mass | |
|---|---|---|---|---|
| Column # | recovered (uL) | (mg/mL) | recovered (mg) | % recovered |
| 1 | 120 | 0.44 | 0.053 | 66 |
| 2 | 125 | 0.54 | 0.068 | 84 |
| 3 | 128 | 0.46 | 0.059 | 74 |
| 4 | 133 | 0.48 | 0.064 | 80 |
| 5 | 130 | 0.43 | 0.056 | 70 |
| 6 | 121 | 0.43 | 0.052 | 65 |
| 7 | 126 | 0.47 | 0.059 | 74 |
| 8 | 119 | 0.53 | 0.063 | 79 |
| 9 | 111 | 0.49 | 0.054 | 68 |
| 10 | 114 | 0.56 | 0.064 | 80 |
| 11 | 98 | 0.61 | 0.060 | 75 |
| 12 | 125 | 0.52 | 0.065 | 81 |
| Ave | 121 | 0.50 | 0.060 | 75 |
| SD | 10 | 0.06 | 0.005 | 6 |
| % CV | 7.9 | 11.3 | 8.5 | 8 |

Performance was enhanced when the pause time between processing the conditioning buffer and addition sample was more carefully controlled. The experiment was repeated and the pause was reduced to 15 minutes from 20 minutes (Table 6).

TABLE 6
Reduce conditioning pause

| | Vol. | [IgG] | Mass | |
|---|---|---|---|---|
| Column # | recovered (μL) | (mg/mL) | recovered (mg) | % recovered |
| 1 | 122 | 0.49 | 0.060 | 75 |
| 2 | 119 | 0.50 | 0.060 | 74 |
| 3 | 122 | 0.50 | 0.061 | 76 |
| 4 | 119 | 0.54 | 0.064 | 80 |
| 5 | 122 | 0.48 | 0.059 | 73 |
| 6 | 123 | 0.51 | 0.063 | 78 |
| Ave | 121 | 0.50 | 0.061 | 76 |
| SD | 2 | 0.02 | 0.002 | 3 |
| % CV | 1.4 | 4.1 | 3.5 | 4 |

### Example 8

### Gel Filtration Columns for Use in Size Exclusion Chromatography

Gel filtration columns were tested for the ability to separate molecules in a complex sample based upon molecular weight and shape. In some instances, agglomeration was simulated by use of large molecules. Gel filtration columns were manufactured containing four different types of resin, GE Sephadex S-200, GE Sephadex S-300, ToyoPearl HW-55F, and GE Superose 12 Prep. Samples containing standard proteins of varying molecular weights were used to measure the separation characteristics of each resin. For all experiments, the columns were made following the standard manufacturing procedure and contained resin beds of 600 μL, 800 μL, or 1000 μL. The columns were equilibrated and conditioned as per Example 2. 100 μL of sample of varying protein composition was loaded from the top of each column and the flow through fraction was collected. Twelve to fourteen 50-μL chaser fractions were collected and analyzed by either UV spectroscopy or HPLC generate a chromatogram.

The standard molecules used in this study were the following:

| Name | Size (MW) |
|---|---|
| Protein X | 350,000 |
| Human immunoglobulin G (hIgG) | 150,000 |
| Bovine serum albumin (BSA) | 67,000 |
| DNPglutamate | 313 |

The high molecular weight Protein X was tested along with the low molecular weight protein, BSA using gel filtration columns containing 600 μL Sephadex S-200 (Table 7). The BSA was releasing early from the column suggesting that the column was either over loaded with BSA or that the BSA was agglomerating. This was determined by comparison with the elution profile of a small molecular weight molecule, DNP-glutamate, which represents a late elution typical of a small molecule. The elution profile of a lower concentration of BSA was tested in addition to the columns conditioned and chased with different a buffer that promoted denaturation, urea, or with a buffer that contained surfactant, Tween-20.

TABLE 7
Detection of molecules after processing in columns
containing 600 μL GE Sephadex S-200

| | | | | 0.7 mg/mL | | |
|---|---|---|---|---|---|---|
| | | 5 mg/mL | 0.7 mg/mL | BSA in PBS, | 3.6 mg/mL | |
| Fraction | Protein X | BSA in | BSA in | 0.05% | BSA in | DNP- |
| # | detection | PBS | PBS | Tween-20 | Urea | glutamate |
| 1 | | | | | | |
| 2 | | | | | | |
| 3 | | | | | | |
| 4 | | | | | | |
| 5 | | | | | + | |
| 6 | + | + | + | + | + | |
| 7 | + | + | + | + | + | |
| 8 | | + | + | + | + | |
| 9 | | + | | | | |
| 10 | | | | | | |
| 11 | | | | | | + |
| 12 | | | | | | + |
| 13 | | | | | | |
| 14 | | | | | | |

In addition to the Sephadex S-200, three other resins were evaluated for the ability to separate samples containing molecules of different molecular weights (Tables 8 and 9).

TABLE 8
Detection of molecules after processing in columns containing GE Sephadex S-300

| | 600 μL resin bed volume | | 800 μL resin bed volume | | 1000 μL resin |
|---|---|---|---|---|---|
| | 0.04 mg/mL | 0.7 mg/mL BSA | | 0.9 mg/mL | bed volume |
| | Protein X in PBS, | in PBS, 0.05% | 0.04 mg/mL | BSA in | 0.8 mg/mL BSA |
| Fraction # | 0.05% Tween-20 | Tween-20 | Protein X in PBS | PBS | in PBS |
| 1 | | | | | |
| 2 | | | | | |
| 3 | | | | | |
| 4 | | | | | |
| 5 | | | | | |
| 6 | + | | | + | |
| 7 | + | + | | + | |
| 8 | + | + | + | + | |
| 9 | | + | + | | |
| 10 | | + | | | |
| 11 | | | | | + |
| 12 | | | | | + |
| 13 | | | | | + |
| 14 | | | | | + |

TABLE 9
Detection of Protein X after processing in columns
containing 600 μL HW-55F or Superose 12

| Fraction # | HW-55F | Superose 12 |
|---|---|---|
| 1 | | |
| 2 | | |
| 3 | | |
| 4 | | |
| 5 | | |
| 6 | + | |
| 7 | + | + |
| 8 | + | + |
| 9 | | + |
| 10 | | |
| 11 | | |
| 12 | | |
| 13 | | |
| 14 | | |

### Example 9

### Gel Filtration Columns for Separation of Nucleic Acid Monomers from Oligonucleotides

Nucleic acids including but not limited to DNA, RNA, DNA/RNA hybrids and nucleic acids containing nucleotide analogs and modifications will be purified of free nucleotides, free labels, salts and other small molecules by gel filtration columns. Additionally, buffer exchange is often required for enzymatic reaction compatibility. Oligonucleotides of different composition and different lengths will be mixed with a small fluorescent dye. These samples will be processed by 600 μL gel filtration columns equilibrated in PBS buffer. 100-μL samples will be applied to the columns and the flow-through will be collected. Next, 100 μL of PBS will be applied to the top of the column and the flow through will be collected in a separate, clean tube. This fractionation will continue for seven more fractions of 100 μL PBS. Sample fractions will be analyzed by UV spectroscopy and the nucleic acid recovery will be measured by absorbance at 260 nm. The contaminating dye will be measured at the appropriate absorbance and the conditions for best nucleic acid recovery and dye removal will be determined.

### Example 10

### Obtaining Flow and Performance Consistency from Gel Filtration Columns

The construction of gel filtration columns is critical to the flow rate. If the resin is over packed, then flow rates will be slowed considerably. If there is a gap between the top frit and the resin bed, then an air bubble will be trapped when fluid is introduced to the top of the column and no flow will occur.

A set of columns must contain the same volume of resin to flow consistently. Several salts were tested to raise the density of the resin slurry to maintain a consistent suspension. The control slurry consisted of 2 g Sephadex G25 resin brought up to 20 mL with a 0.01% sodium azide solution. Another identical slurry was made except it was supplemented with 24 g cesium chloride. The addition of cesium chloride resulted in slurry staying in suspension with less agitation. 24 gel-filtration columns were packed with 200 μL of each resin and washed with 6 mL of 0.01% sodium azide. The flow characteristics of these packed bed columns was measured before the top frits were placed above the resin bed. 700 μL 0.01% sodium azide was added to the top of each column and the time for the fluid to completely enter the resin bed was recorded (Table 10). This experiment was done in triplicate. The results of this showed that columns manufactured with cesium chloride flowed slightly slower (11 minutes, 38 seconds on average) than those made without (9 minutes 50 seconds on average).

The impact of the top frit was tested by taking the columns manufactured described above and adding the top screen at various heights. First, the 24 columns manufactured with cesium chloride had top frits inserted to where the top frit was just touching the resin bed. Slight compression of the resin bed may have occurred but it was minimal (<1 mm). Again, 700 μL of 0.1% sodium azide was added to the top of the columns and the time for fluid to completely flow through the resin bed was recorded (Table 11). This experiment was run in triplicate. The mean flow time for these columns was 12 minutes, 0 seconds, which was slightly longer than the columns without inserts. Columns #9 and #17 had a slight gap between the top of the resin bed and the top frit. This was noticed after the first trial, which is why they did not flow. The top frits were re-seated prior to the next run by having the frit just touch the resin. The data from these two columns was not included in the mean flow time calculation. To test how compression of the top screen affects flow, these columns were stressed by pushing the top frit down approximately 1 mm. Four measurements for the time for 700 μL of 0.1% sodium azide to completely flow through the resin bed was recorded (Table 11). The average flow time for these columns was 15 minutes and 13 seconds. The impact of compressing the top frit an additional 1 mm resulted in slowing the processing time to 21 minutes and 45 seconds (Table 12).

To test how a gap affects the flow of fluid through the resin bed, 24 columns that were manufactured without CsCl, described above, were used to test inserts of either 1.5 mm above the resin bed or with less than 1 mm of compression (Table 13). The result of a less than 1 mm compression resulted in a flow processing time of 11 minutes, 31 seconds.

A final variation of the top screen was tested to attempt to alleviate the compression of the resin bed. Columns 9-16 manufactured without CsCl were used to test frit screens with a slit cut through the diameter. When these frits were placed 1.5 mm above the resin bed, there is no flow. When the frits were re-seated to compress the resin bed by <1 mm, then the mean flow was 11 minutes, 52 seconds. Then the compression increased to 1 mm, the flow was prolonged to 12 minutes, 28 seconds.

TABLE 10
Columns manufactured with and without cesium chloride in the resin slurry

| | Slurry composition: 0.01% sodium azide | | | | Slurry composition: 0.01% sodium azide, CsCl | | | |
|---|---|---|---|---|---|---|---|---|
| | Time to | Time to | Time to | | Time to | Time to | Time to | |
| | process | process | process | Ave. | process | process | process | Ave. |
| | 700 μL-1 | 700 μL-2 | 700 μL-3 | processing | 700 μL-1 | 700 μL-2 | 700 μL-3 | processing |
| Column # | (min.) | (min.) | (min.) | time (min.) | (min.) | (min.) | (min.) | time (min.) |
| 1 | 8.75 | 8.75 | 9.00 | 8.83 | 10.00 | 10.25 | 10.50 | 10.25 |
| 2 | 11.50 | 10.75 | 10.50 | 10.92 | 10.75 | 10.75 | 10.50 | 10.67 |
| 3 | 10.25 | 10.25 | 10.25 | 10.25 | 12.00 | 12.00 | 12.25 | 12.08 |
| 4 | 9.75 | 9.25 | 8.75 | 9.25 | 11.00 | 10.75 | 11.00 | 10.92 |
| 5 | 9.75 | 9.25 | 9.25 | 9.42 | 12.00 | 12.00 | 12.25 | 12.08 |
| 6 | 9.75 | 9.25 | 10.25 | 9.75 | 10.25 | 10.25 | 10.25 | 10.25 |
| 7 | 10.25 | 9.75 | 9.75 | 9.92 | 10.25 | 10.25 | 11.50 | 10.67 |
| 8 | 9.25 | 9.75 | 9.75 | 9.58 | 11.00 | 10.75 | 11.50 | 11.08 |
| 9 | 9.25 | 10.00 | 9.00 | 9.42 | 12.50 | 13.00 | 13.00 | 12.83 |
| 10 | 9.75 | 10.50 | 9.50 | 9.92 | 11.00 | 11.50 | 11.50 | 11.33 |
| 11 | 10.25 | 10.50 | 9.50 | 10.08 | 11.00 | 11.50 | 11.50 | 11.33 |
| 12 | 9.75 | 10.00 | 9.75 | 9.83 | 11.00 | 11.50 | 11.50 | 11.33 |
| 13 | 10.25 | 10.50 | 9.75 | 10.17 | 12.25 | 12.25 | 12.50 | 12.33 |
| 14 | 10.50 | 10.50 | 10.25 | 10.42 | 12.50 | 13.00 | 13.25 | 12.92 |
| 15 | 9.50 | 9.25 | 9.50 | 9.42 | 11.50 | 12.25 | 12.25 | 12.00 |
| 16 | 9.25 | 9.75 | 10.25 | 9.75 | 11.50 | 12.25 | 12.25 | 12.00 |
| 17 | 8.50 | 9.00 | 9.25 | 8.92 | 10.25 | 10.00 | 10.75 | 10.33 |
| 18 | 10.00 | 10.25 | 10.00 | 10.08 | 11.50 | 11.25 | 11.25 | 11.33 |
| 19 | 10.00 | 10.00 | 10.25 | 10.08 | 11.50 | 13.00 | 12.75 | 12.42 |
| 20 | 10.00 | 10.00 | 10.25 | 10.08 | 11.50 | 12.50 | 12.75 | 12.25 |
| 21 | 9.50 | 10.25 | 9.75 | 9.83 | 11.50 | 11.75 | 11.75 | 11.67 |
| 22 | 10.25 | 10.25 | 9.75 | 10.08 | 10.50 | 11.50 | 10.75 | 10.92 |
| 23 | 10.25 | 10.00 | 9.75 | 10.00 | 11.50 | 13.25 | 12.75 | 12.50 |
| 24 | 9.50 | 10.00 | 9.75 | 9.75 | 10.50 | 11.25 | 11.75 | 11.17 |
| Ave. | 9.82 | 9.91 | 9.74 | 9.82 | 11.22 | 11.61 | 11.75 | 11.53 |

TABLE 11
Columns manufactured with top frit insert screens

| | No compression of resin bed | | | | 1 mm compression of resin bed | | | | |
|---|---|---|---|---|---|---|---|---|---|
| | Time to | Time to | Time to | Ave. | Time to | Time to | Time to | Time to | Ave. |
| | process | process | process | processing | process | process | process | process | processing |
| | 700 μL-1 | 700 μL-2 | 700 μL-3 | time | 700 μL-1 | 700 μL-2 | 700 μL-3 | 700 μL-4 | time |
| Column # | (min.) | (min.) | (min.) | (min.) | (min.) | (min.) | (min.) | (min.) | (min.) |
| 1 | 10.50 | 12.25 | 11.75 | 11.50 | 12.25 | 12.50 | 13.50 | 13.25 | 12.88 |
| 2 | 9.50 | 10.50 | 11.75 | 10.58 | 14.50 | 15.00 | 14.75 | 15.00 | 14.81 |
| 3 | 12.00 | 13.25 | 13.75 | 13.00 | 13.00 | 14.00 | 13.50 | 13.75 | 13.56 |
| 4 | 10.25 | 11.50 | 11.75 | 11.17 | 14.00 | 14.25 | 14.75 | 15.00 | 14.50 |
| 5 | 11.00 | 12.75 | 13.25 | 12.33 | 14.00 | 15.00 | 14.75 | 14.75 | 14.63 |
| 6 | 10.00 | 12.25 | 11.75 | 11.33 | 12.75 | 13.50 | 13.25 | 14.00 | 13.38 |
| 7 | 10.00 | 11.00 | 11.75 | 10.92 | 13.75 | 15.00 | 14.75 | 14.75 | 14.56 |
| 8 | 10.00 | 11.00 | 10.75 | 10.58 | 15.50 | 15.50 | 16.50 | 16.75 | 16.06 |
| 9 | No Flow | 13.75 | 14.00 | 13.88 | 13.25 | 14.25 | 13.50 | 14.50 | 13.88 |
| 10 | 11.50 | 12.00 | 12.25 | 11.92 | 13.25 | 14.25 | 13.50 | 14.00 | 13.75 |
| 11 | 11.50 | 12.00 | 12.25 | 11.92 | 17.50 | 18.25 | 18.25 | 18.50 | 18.13 |
| 12 | 11.50 | 12.00 | 12.25 | 11.92 | 17.00 | 17.50 | 14.25 | 14.00 | 15.69 |
| 13 | 12.00 | 12.00 | 12.25 | 12.08 | 15.25 | 15.75 | 16.00 | 15.75 | 15.69 |
| 14 | 12.50 | 12.75 | 13.50 | 12.92 | 12.50 | 13.25 | 14.00 | 14.50 | 13.56 |
| 15 | 10.25 | 10.25 | 11.00 | 10.50 | 14.50 | 16.00 | 16.25 | 16.25 | 15.75 |
| 16 | 10.25 | 10.25 | 11.00 | 10.50 | 14.75 | 14.25 | 14.25 | 14.25 | 14.38 |
| 17 | No Flow | 13.50 | 15.50 | 14.50 | 12.25 | 13.75 | 12.75 | 13.50 | 13.06 |
| 18 | 11.00 | 11.50 | 12.00 | 11.50 | 17.50 | 17.75 | 18.25 | 18.25 | 17.94 |
| 19 | 12.00 | 12.50 | 12.50 | 12.33 | 14.00 | 14.75 | 15.25 | 15.25 | 14.81 |
| 20 | 17.00 | 15.75 | 14.75 | 15.83 | 15.75 | 16.50 | 17.00 | 16.50 | 16.44 |
| 21 | 11.00 | 11.75 | 11.30 | 11.35 | 17.25 | 18.75 | 18.25 | 18.50 | 18.19 |
| 22 | 9.50 | 11.50 | 12.00 | 11.00 | 13.50 | 14.75 | 15.25 | 16.50 | 15.00 |
| 23 | 12.25 | 12.75 | 13.75 | 12.92 | 16.00 | 18.50 | 17.50 | 18.00 | 17.50 |
| 24 | 11.75 | 11.25 | 11.25 | 11.42 | 16.00 | 17.25 | 17.50 | 17.00 | 16.94 |
| Ave. | 11.24 | 12.08 | 12.42 | 12.00 | 14.58 | 15.43 | 15.31 | 15.52 | 15.21 |

TABLE 12
Compressing the resin bed by 2 mm

| | Time to process | Time to process | Ave. processing |
|---|---|---|---|
| Column # | 700 μL-1 (min.) | 700 μL-2 (min.) | time (min.) |
| 1 | 19.00 | 18.25 | 18.63 |
| 2 | 17.00 | 15.75 | 16.38 |
| 3 | 14.00 | 14.00 | 14.00 |
| 4 | 25.00 | 24.75 | 24.88 |
| 5 | 21.00 | 20.00 | 20.50 |
| 6 | 23.75 | 23.50 | 23.63 |
| 7 | 25.00 | 23.50 | 24.25 |
| 8 | 25.00 | 23.75 | 24.38 |
| 9 | 19.25 | 19.75 | 19.50 |
| 10 | 20.00 | 19.75 | 19.88 |
| 11 | 23.25 | 24.00 | 23.63 |
| 12 | 20.50 | 20.50 | 20.50 |
| 13 | 26.00 | 26.00 | 26.00 |
| 14 | 17.25 | 16.50 | 16.88 |
| 15 | 27.00 | 26.50 | 26.75 |
| 16 | 27.00 | 26.50 | 26.75 |
| 17 | 21.25 | 20.25 | 20.75 |
| 18 | 28.00 | 28.00 | 28.00 |
| 19 | 24.00 | 21.50 | 22.75 |
| 20 | 22.50 | 21.50 | 22.00 |
| 21 | 23.50 | 21.75 | 22.63 |
| 22 | 19.50 | 18.50 | 19.00 |
| 23 | 18.00 | 17.00 | 17.50 |
| 24 | 24.00 | 21.75 | 22.88 |
| Ave. | | | |

TABLE 13
Minimal compression of the resin bed

| | 1.5 mm gap | | | |
|---|---|---|---|---|
| | between resin | Compression of resin bed by <1 mm | | |
| | bed and frit | Time to | Time to | |
| | Time to | process | process | |
| | process 700 μL- | 700 μL-1 | 700 μL-2 | Ave. processing |
| Column # | 1 (min.) | (min.) | (min.) | time (min.) |
| 1 | No Flow | 11.75 | 12.25 | 12.00 |
| 2 | No Flow | 12.75 | 14.00 | 13.38 |
| 3 | No Flow | 12.00 | 12.50 | 12.25 |
| 4 | No Flow | 14.50 | 14.75 | 14.63 |
| 5 | No Flow | 12.00 | 12.25 | 12.13 |
| 6 | No Flow | 14.00 | 13.50 | 13.75 |
| 7 | No Flow | 11.75 | 11.75 | 11.75 |
| 8 | No Flow | 11.75 | 12.25 | 12.00 |
| Ave. | | 12.56 | 12.91 | 12.73 |

TABLE 14
Frit with a slit through the diameter of the screen

| | | | | | Compression |
|---|---|---|---|---|---|
| | 1.5 mm gap | | | | of resin bed |
| | between resin | Compression of resin bed by <1 mm | | | by 1 mm |
| | bed and frit | Time to | Time to | | Time to |
| | Time to | process | process | | process |
| | process 700 μL-1 | 700 μL-1 | 700 μL-2 | Ave. processing | 700 μL-1 |
| Column # | (min.) | (min.) | (min.) | time (min.) | (min.) |
| 9 | No Flow | 10.75 | 11.25 | 11.00 | 10.75 |
| 10 | No Flow | 11.75 | 11.50 | 11.63 | 11.00 |
| 11 | No Flow | 11.50 | 12.50 | 12.00 | 12.25 |
| 12 | No Flow | 10.50 | 11.25 | 10.88 | 12.25 |
| 13 | No Flow | 12.00 | 11.75 | 11.88 | 12.25 |
| 14 | No Flow | 11.75 | 11.50 | 11.63 | 12.25 |
| 15 | No Flow | 12.00 | 11.75 | 11.88 | 16.50 |
| 16 | No Flow | 10.75 | 11.75 | 11.25 | 12.50 |
| Ave. | | 11.38 | 11.66 | 11.52 | 12.47 |

### Example 11

### Gel Filtration Column Back Pressures

Gel filtration columns were packed with 200 μL, 600 μL or 1 mL of different gel filtration media. Columns were pumped with water at a flow rate of either 0.5 mL/minute or 1 mL/minute and the back pressure was measured. The flow rate is linearly proportional to pressure with a slope of 1. The results are shown in Table 15.

TABLE 15

| | Resin bed | Back pressure | Back pressure |
|---|---|---|---|
| Resin type | vol. (μL) | 1 mL/minute (PSI) | 0.5 mL/minute (PSI) |
| Sephadex G25 | 200 | 0.4 | |
| Sephadex G25 | 200 | 0.5 | |
| Sephadex G25 | 600 | 1.0 | |
| Sephadex G25 | 1000 | 0.8 | |
| Sephadex G25 | 1000 | 0.7 | |
| Superose 12 | 600 | | 4.1 |
| Superose 12 | 600 | | 3.5 |
| Toyopearl HW55 | 600 | 3.7 | |
| Toyopearl HW55 | 600 | 4.0 | |
| Sephacryl S200 | | | 1.9 |
| Sephacryl S200 | | | 2.5 |
| Sephacryl S300 | | | 2.3 |

While the invention has been described in connection with specific embodiments thereof, it will be understood that it is capable of further modifications and this application is intended to cover and variations, uses, or adaptations of the invention that follow, in general, the principles of the invention, including such departures from the present disclosure as come within known or customary practice within the art to which the invention pertains and as may be applied to the essential features hereinbefore set forth. Moreover, the fact that certain aspects of the invention are pointed out as preferred embodiments is not intended to in any way limit the invention to such preferred embodiments.

What is claimed is:

1. A method for purifying a material from a sample solution using gravity flow chromatography comprising the steps of:
a. providing at least one chromatography column, wherein each column is comprised of
i) a column body having an open upper end, an open lower end, and an open channel between the upper and lower end of the column body,
ii) a bottom frit extending across the open channel,
iii) a packed bed of chromatography medium positioned above the bottom frit, wherein the diameter of each column is within the range of about 12 to about 100 mm²;
b. introducing a sample solution into each column;
c. allowing the sample solution to pass through the column by gravity flow until the flow pauses;
d. introducing an elution liquid into each column;
e. allowing the elution liquid to pass through the column by gravity flow until the flow pauses;
f optionally, repeating steps (d) and (e) at least once;
g. collecting the purified material;
h. optionally, repeating steps (d), (e) and (g).

2. The method of claim 1, wherein the method is automated and steps (b) and (d) are performed by a liquid handler.

3. The method of claim 1, wherein the method is manual and steps (b) and (d) are performed with a pipette.

4. The method of claim 1, wherein the column body is comprised of a modified pipette tip.

5. The method of claim 1, wherein the column body is further comprised of a top frit positioned above the packed bed of chromatography medium.

6. The method of claim 1, wherein prior to step (g), a collection plate is provided and step (g) is performed by touching the open lower end of the columns to the walls of the collection plate wells.

7. The method of claim 1, where in the volume of purified material is in the range of 5 μl to 600 μl.

8. The method of claim 7, where in the volume of purified material is in the range of 20 μl to 90 μl.

9. The method of claim 1, wherein the method is performed on a plurality of columns the volume of purified material obtained from the columns has a coefficient of variation of less than 20.

10. The method of claim 1, wherein the distance between the centers of the columns is in the range of about 4.5 mm to about 9.0 mm.

11. The method of claim 10 wherein each column is integrated into a well of a deep-well plate.

12. The method of claim 10, wherein the method is automated and steps (b) and (d) are performed by a liquid handler.

13. The method of claim 10, wherein the method is manual and steps (b) and (d) are performed with a pipette.

14. The method of claim 10, wherein the column body is comprised of a modified pipette tip.

15. The method of claim 10, wherein the column body is further comprised of a top frit positioned above the packed bed of chromatography medium.

16. The method of claim 10, wherein prior to step (g), a collection plate is provided and step (g) is performed by touching the open lower end of the columns to the walls of the collection plate wells or vials.

17. The method of claim 10, where in the volume of purified material is in the range of 5 μl to 600 μl.

18. The method of claim 17, where in the volume of purified material is in the range of 20 μl to 90 μl.

19. A plurality of chromatography columns, wherein each column is comprised of
a. a column body having an open upper end, an open lower end, and an open channel between the upper and lower end of the column body;
b. a bottom frit extending across the open channel;
c. a packed bed of medium positioned above the bottom frit; and
d. a top frit extending across the open channel,
wherein the distance between the centers of the columns is within the range of about 4.5 to about 9.0 mm.
